(19) United States
(12) Patent Application Publication
(10) Pub. No.: US 2022/0261990 A1
(43) Pub. Date: Aug. 18, 2022

(54) MACHINE LEARNING FOR EARLY DETECTION OF CELLULAR MORPHOLOGICAL CHANGES

(71) Applicant: ViQi, Inc., Santa Barbara, CA (US)

(72) Inventors: Ilya Goldberg, Santa Barbara, CA (US); Dmitry Fedorov, Santa Barbara, CA (US); Christian A. Lang, Santa Barbara, CA (US); Kristian Kvilekval, Santa Barbara, CA (US); Katherine Yeung, Santa Barbara, CA (US); Henry Rupert Dodkins, Santa Barbara, CA (US)

(21) Appl. No.: 17/650,067

(22) Filed: Feb. 4, 2022

Related U.S. Application Data
(60) Provisional application No. 63/228,093, filed on Jul. 31, 2021.
(60) Provisional application No. 63/146,541, filed on Feb. 5, 2021.

(51) Int. Cl. G06T 7/00; G06V 20/69; G06N 20/00; G16H 50/20

(52) U.S. Cl. CPC G06T 7/0012; G06V 20/69; G06N 20/00; G06T 2207/10056; G06T 2207/30072; G06T 2207/20084; G16H 50/20

(57) ABSTRACT

Methods and systems for machine learning are disclosed for early detection of morphological changes in cell condition of biological cells. In one disclosed embodiment, the development of vaccines and anti-virals are sped up using machine learning to identify viral plaques earlier than can be detected using human observation alone. In the disclosed embodiment, detecting morphological changes in virus-infected cells can be made before plaques caused by cell death are observable (typical cell death in 2-14 days). Machine learning brings high-content/high-throughput techniques to the study of virology for the development of novel anti-viral compounds. Machine learning can also be used to characterize the effectiveness of novel anti-viral compounds on rapidly mutating viral strains, such as influenza and SARS-CoV-2.

### CROSS-REFERENCE TO RELATED APPLICATIONS

This patent application claims the benefit of U.S. Provisional Patent Application No. 63/228,093 titled MACHINE LEARNING FOR EARLY DETECTION OF CELLULAR MORPHOLOGICAL CHANGES filed on Jul. 31, 2021 by inventors Ilya Goldberg et al.; and also claims the benefit of U.S. Provisional Patent Application No. 63/146,541 titled MACHINE LEARNING FOR EARLY DETECTION OF CELLULAR MORPHOLOGICAL CHANGES filed on Feb. 5, 2021 by inventors Ilya Goldberg et al., both of which are incorporated herein by reference for all intents and purposes.

### GOVERNMENT LICENSE RIGHTS

This invention was made with government support under grant award number 2029707 awarded by the National Science Foundation. The government has certain rights in the invention.

### FIELD

The disclosed embodiments relate generally to machine learning about biological cells from digital images for early detection of changes in cellular structure, such as the early detection of COVID-19 plaques in cells.

### BACKGROUND

A plaque assay is one of the most important assays in virology. It measures the number of infectious viral particles in a sample by observing the effects of infection on a culture of susceptible cells. Currently, it takes about two to fourteen days to process a plaque assay because several rounds of infection are necessary to ensure an accurate read-out. It is desirable to reduce the time to get results of infectivity.

Observing changes over an extended period of time in biological cells is difficult to do with the human eye, even when aided by a microscope. The changes to biological cells may be so subtle and can occur amidst a noisy environment such that the changes in the biological cells can be overlooked by a person studying hundreds of cells under a microscope. It is desirable to improve the capture and analysis of changes in numerous biological cells over periods of time.

### BRIEF SUMMARY

The embodiments are summarized by the claims that follow below.

### BRIEF DESCRIPTION OF THE DRAWINGS

This patent or application file contains at least one drawing executed in color. Copies of this patent or patent application publication with color drawing(s) will be provided by the United States Patent and Trademark Office upon request and payment of the necessary fee.

FIG. 1A is a block diagram of a client-server computer system with multiple client computers communicating with one or more computer servers in a server center (or the cloud) over a computer network, such as a wide area network of the internet, and the services offered.

FIG. 1B illustrates a block diagram of a biological cell analysis system implementing an early detection system for cellular morphological changes.

FIG. 2A illustrates a user interface window generated by the system including a slide viewer illustrating a brightfield image of biological cells (center) and annotations (right panel) of some of the cells.

FIG. 2B illustrates the detail of the right-side panel of the user interface shown in FIG. 2A.

FIG. 3 illustrates a user interface window generated by the system with an example output of machine learning classification of classified cells for detecting morphological changes in brightfield images.

FIG. 4A illustrates another user interface viewer window provided by the system.

FIG. 4B illustrates the viewer window of brightfield image of cells overlaid with the classification results from classification algorithms using a generated AI model.

FIG. 5A illustrates workflow diagrams to compare a traditional assay workflow for a standard viral plaque assay with that of an AI enhanced assay workflow, including a machine learning/model training phase with brightfield images of cells with known cell conditions and a diagnostic phase with cell classification of cells to be tested for their cell condition by an AI model.

FIG. 5B illustrates a workflow diagram of the two phases, training and classification for AI powered cell assays according to embodiments of the invention.

FIG. 5C illustrates a workflow diagram and timeline comparing a traditional plaque assay with an AI enhanced assay.

FIG. 5D illustrates image capture of digital images of wells in a plate with infected biological cells and the formation of rectangular tiles of pixels from a digital image.

FIG. 5E illustrates a validation chart with plots to compare infectivity determined with an AI model and machine learning algorithms versus infectivity determined with traditional assay methods.

FIG. 6 illustrates a block diagram of a client-server computer system with multiple client computers communicating with one or more computer servers in a server center (or the cloud) over a computer network, such as a wide area network of the internet.

FIG. 7 illustrates a block diagram of a computer system for use as a server computer and client computers (devices) in the system shown in FIG. 6.

### DETAILED DESCRIPTION

Infectious disease will likely always be around us. The ability to react quickly to novel virus mutations is key to handling dangerous outbreaks and protecting vulnerable populations. For example, experts have warned that as with influenza, we can expect more outbreaks of the COVID-19 virus due to seasonal variations. Therefore, rapid and accurate identification of viral infections are critical to our future.

The disclosed embodiments can speed the development of vaccines and antivirals, gene therapy vectors, and oncolytic viral therapies using machine learning to develop a rapid automated infectivity assay based on identifying virus-infected cells in brightfield microscopy within hours of infection. Machine learning can detect cell morphologies associated with virus infection even when they cannot be detected by human observers. The disclosed embodiments do not rely on detecting cell death after multiple rounds of infection like in traditional plaque or TCID50 assays, instead relying on much earlier infection events. The use of brightfield microscopy obviates cell fixation and staining which allows the technique to be more easily automated as well as making it faster and more cost effective. Besides brightfield images, other digital images of virus-infected cells can be used, such as darkfield images from darkfield microscopy, phase contrast images from phase contrast microscopy, and differential interference contrast (DIC) images from DIC microscopy.

Benefits of the disclosed embodiment include:
Shorten viral assays from 2-14 days to hours—no multiple rounds of infection needed.
Ability to automate and scale testing.
Increased throughput with standard High Content Screening (HCS) devices.
Reduced materials, reagent and compound costs—No agarose overlays or fixation necessary.
No antibodies or transgenic virus necessary for early detection.
Elimination of error-prone manual results through objective, quantitative readouts.
Reduced cost per assay through miniaturization.
Reduced time for training of staff to perform manual plaque assays.
For specialized testing in BSL3/4 labs, exponential savings of training and time.
Potential to radically transform workflow and research for more rapid turns, parallel testing and more rapid screening.
Increase responsiveness to another outbreak, variants of COVID-19 or future pandemics.

Other embodiments of the invention, such as cloud-based image processing and machine learning can bring high content screening (HCS) techniques to the study of virology to increase the pace of development of novel anti-viral compounds or characterize their effectiveness on rapidly mutating viral strains such as influenza and SARS-CoV-2.

Rapid accurate identification of a virus can be used to implement effective means of contact tracing, quarantine, and the deployment of effective antiviral agents.

At the same time, HCA (High Content Analysis) has proved invaluable to early drug discovery programs. The disclosed embodiments can produce rapid turn-around and screening of potential vaccines and anti-virals as well as potentially automating the search for vaccines and anti-virals.

The ability to detect single infected cells and differentiate viral infection with sufficient accuracy from other types of cellular distress can dramatically alter the time and effort currently required for these types of assays, which can revolutionize the ways that this type of assay is used in virology. Furthermore, biotech and pharmaceutical companies have an immediate interest in new technology enhancing their ability to identify and deliver novel vaccines or anti-virals. The disclosed embodiments can be automated in high-content analysis (HCA) platforms and thus more easily fit into existing drug discovery pipelines. Given the significant cost of drug development, additional subscription fees for a machine learning platform are negligible.

The disclosed embodiments use artificial intelligence (machine learning) to recognize patterns without the need for human intervention. The broad principals disclosed can be applied to plaque assays as detailed below. Equally, the disclosed embodiments are also applicable to drug dosage response of cells, cell toxicity, and detection of live/dead cell detection without the use of fluorescence labelling.

The disclosed embodiments use machine learning to accelerate testing of anti-viral agents. The disclosed embodiments take a novel approach to viral assays by training modern artificial intelligence (AI) models (AIs) to detect individual infected cells or small clusters of infected cells using brightfield (phase contrast or differential interference contrast) microscopy earlier than they can be detected manually, and without the use of viable stains.

The AI models disclosed herein differentiate infected and uninfected cells based on morphological changes in the cells. The AI models process images of cells in biological samples and can also be referred to herein as imaging artificial intelligence (AI) models. The use of AI obviates the need for several slow elements of the assay protocol, resulting in increased throughput for evaluating vaccines and antiviral drugs. AI can also facilitate automation of high-content analysis (HCA) platforms and thus more easily fit into existing drug discovery pipelines.

Several hurdles were overcome to achieve this goal: (1) the feasibility of brightfield microscopy image acquisition during incubation had to be investigated with respect to ease of integration in existing workflows and image quality for machine learning, (2) machine learning algorithms had to be compared with respect to best fit for the amount and quality of images, and (3) prediction quality had to be comparable to the traditional assay read-out.

The disclosed embodiments extend High-Content Analysis (HCA) to the field of virology by using automated screening to characterize effectiveness of vaccines and compounds on disrupting early stages of viral infection. Automation will permit the screening of many more compounds under many more conditions, leading to faster development of antiviral remedies. This type of plaque assay development is applicable to other cell types and viruses, and so this could also be used in influenza studies, for example. Furthermore, experts have warned that as with influenza we can expect more outbreaks of COVID-19 due to seasonal variations; therefore, rapid identification of effective antiviral agents are critical to our future.

The plaque assay is one of the most important assays in virology. It measures the number of infectious viral particles in a sample by observing the effects of infection on a culture of susceptible cells. This assay counts the number of infectious viral particles in a sample (plaque-forming units or PFUs) which is in contrast to assays based on amplifying genetic material (e.g., rtPCR), which only count the number of viral genomes. The reason this assay is crucial in vaccine and antiviral development is that often these treatment strategies interfere with the viral infection pathway. In fact, an increase in the particle (or genome) to PFU ratio is often used as an indicator of the presence of neutralizing antibodies in a candidate vaccine, or effectiveness of an antiviral drug.

Currently, the typical assay takes two to fourteen days to develop because several rounds of infection are necessary to ensure that there are localized clusters of dead cells defining each plaque indicating an infection, rather than single dead cells which could occur for any number of reasons. The clusters of dead cells are typically detected with an end-point viability stain such as crystal violet.

The disclosed embodiments detect viral plaques, in an automated manner, by training modern artificial intelligence models (AIs) to detect individual infected cells or small clusters of infected cells using brightfield (phase contrast or differential interference contrast) microscopy earlier than they can be detected manually, and without the use of viable stains.

The disclosed embodiments detect individual infected cells, thus obviating the need for several elements of the current protocol that make it slow, e.g., multiple rounds of infection, agarose overlay to limit viral diffusion, and end-point viability staining. The final outcome can be an increased throughput for evaluating vaccines and antiviral drugs.

Modern artificial intelligence models (AIs) have not been previously used to identify individual virally infected cells without labels or stains. The disclosed embodiments can use single-cell image-based assays with AI models to discern differences in cells and tissues that cannot be differentiated by highly trained human observers. Artificial intelligence models (AIs) can readily differentiate sub-cellular patterns that even trained human experts could not with immuno-fluorescence assays on individual cells. Using differential interference contrast (DIC) and phase contrast, artificial intelligence models (AIs) can readily differentiate samples of unstained (stain free) cells and tissues. Human observers cannot do so without labels or stains. Moreover, human observers cannot quickly observe and count samples with large quantities of infected biological cells to determined infection concentrations.

With supervised machine learning, the disclosed embodiments use the best available algorithms applied to the traditional plaque assay that is currently a bottleneck in the search for antiviral therapies. A specific challenge overcome by the disclosed embodiments is the experimental design to collect an appropriate training set of infected cells, in a state prior to them developing visible changes (which are typically lysis and death), but without using stains, markers or other means to highlight the cells undergoing early stages of infection.

Given the rapid growth of COVID-19 cases around the globe and the real danger of subsequent waves of infections in years to come, the timely development of a vaccine or anti-viral treatment is of highest priority. While not resulting in such treatment options directly, disclosed embodiments indirectly accelerate the development of treatments by shortening the development pipeline of cell assays. The disclosed embodiments can provide crucial research tools to drug development experts to more quickly develop drugs to counter infections.

The disclosed embodiments can do more than just applying machine learning approaches to existing plaque assays. The AI models do not just count plaque forming units (PFU). The AI models that are developed can differentiate subcellular patterns and display them in a user interface.

Generally, the disclosed embodiments perform an analysis of a viral assay, generate models to recognize infections cells, and disseminate the resulting AI models rapidly, without requiring a significant amount of capital investment for custom software development related to microscopy image format readers, scalable slide viewers, data processing for machine learning, model evaluation and sharing, and a web-based interface to minimize time-consuming installation and configuration.

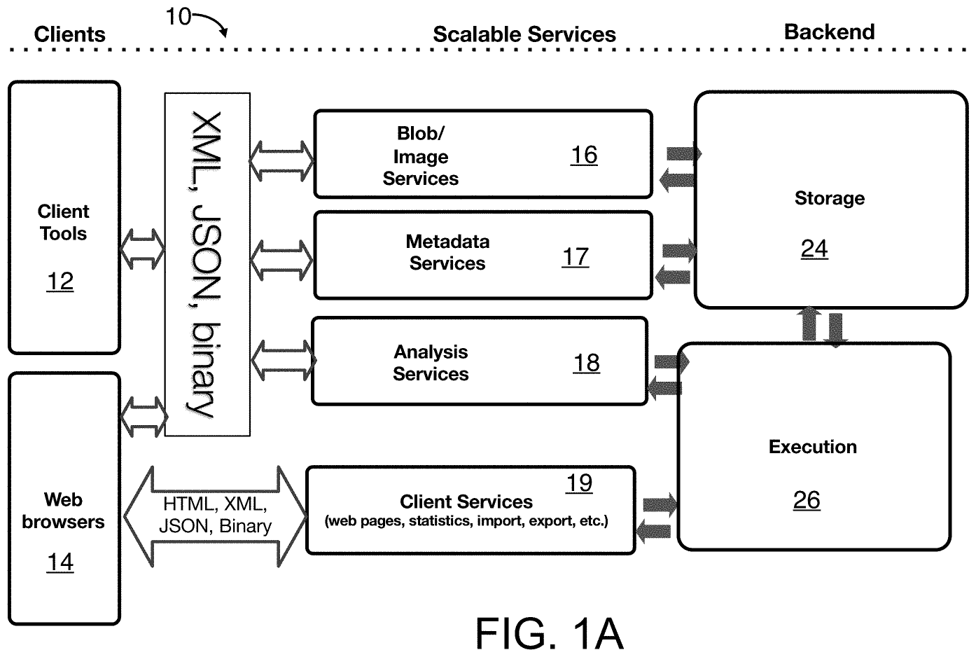

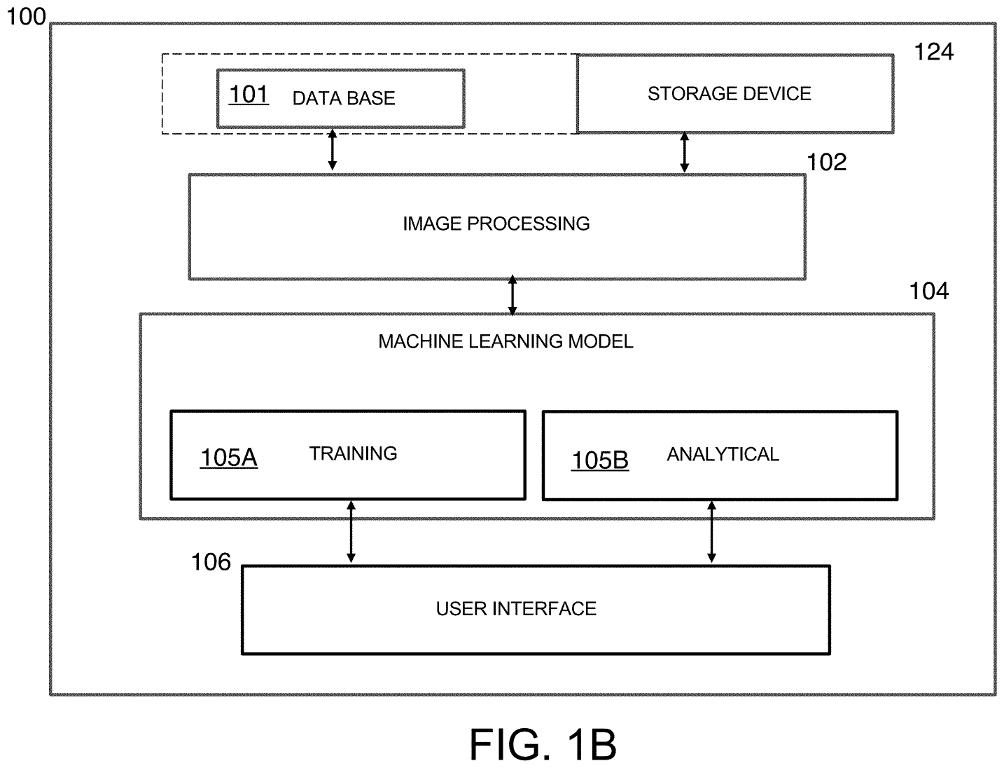

Referring now to FIG. 1A, a web-based scalable image analysis platform 10 is shown. The web-based scalable image analysis platform 10 is usable for a variety of image management and analysis tasks in life sciences. A vast majority of images processed by the web-based scalable image analysis platform are high resolution microscopy images.

The web-based scalable image analysis platform 10, modeled after a BisQue system, can be used to analyze biological cells. BisQue is an open-source image database and analysis system built for biologists working with digital artifacts as a fundamental tool for gathering evidence. The system is built to manage and analyze both data artifacts and metadata for large-scale datasets. BisQue has gained increasing adoption by several large bio-imaging labs world-wide. For example, the CyVerse cyber-infrastructure serves thousands of users using BisQue for its imaging needs.

The web-based scalable image analysis platform 10, being a cloud-based platform, is built with a scalable service-oriented architecture. The web-based scalable image analysis platform 10 provides access to services through web browsers 14 and client tools 12. The web browsers 14 access client services 19 including web pages with viewers and statistics and can be used for general control, such as file import and file export.

Web browsers 14 and client tools 12 can interface to a plurality of micro-services 16-18, each performing a specialized task. A web browser can also act as a client tool (Javascript) and interface to the microservices 16-18 (e.g., running analysis modules from a webpage). A metadata service 17 allows metadata to be stored in storage 24 and queried as nested tag/value documents. Examples for metadata include sample preparation, experimental conditions, imaging parameters, and results. A blob and image service 16 provides access to binary datasets such as images and provides methods to perform pixel operations for example. An analysis service 18 allows running complex analysis modules written in many languages (e.g., Python, Matlab). These analysis modules of the analysis service 18 typically interact with the other services (e.g., to fetch image tiles or metadata records) during computation. The microservices 16-18 and the client services 19 can interface to the backend services of storage services 24 associated with storage devices and execution services 26 associated with computer processors or compute clusters.

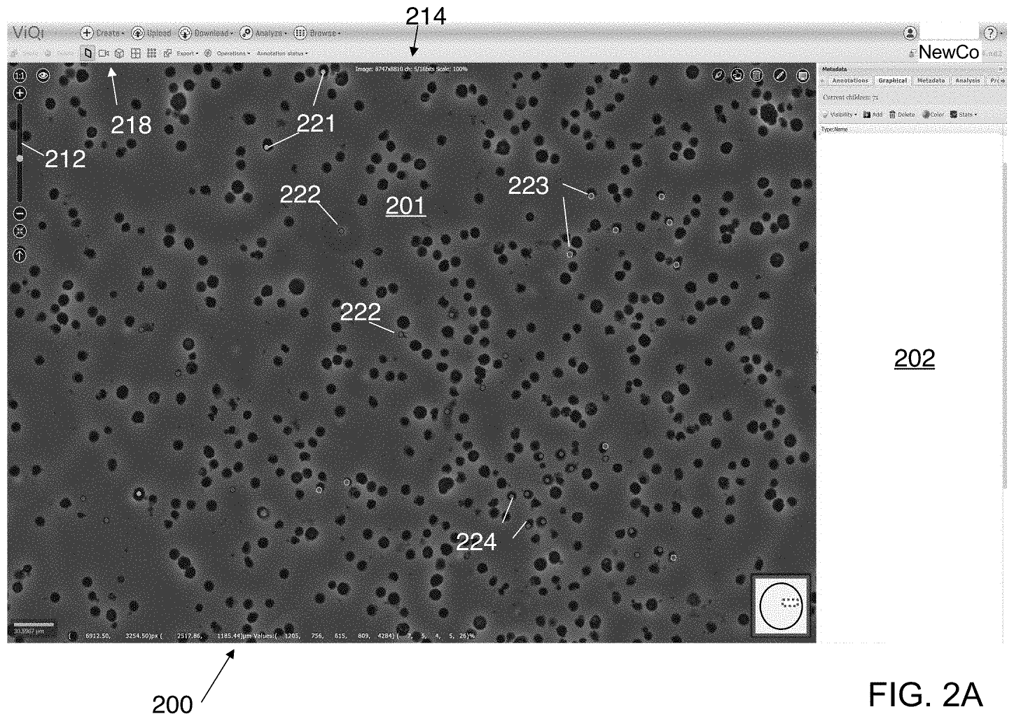

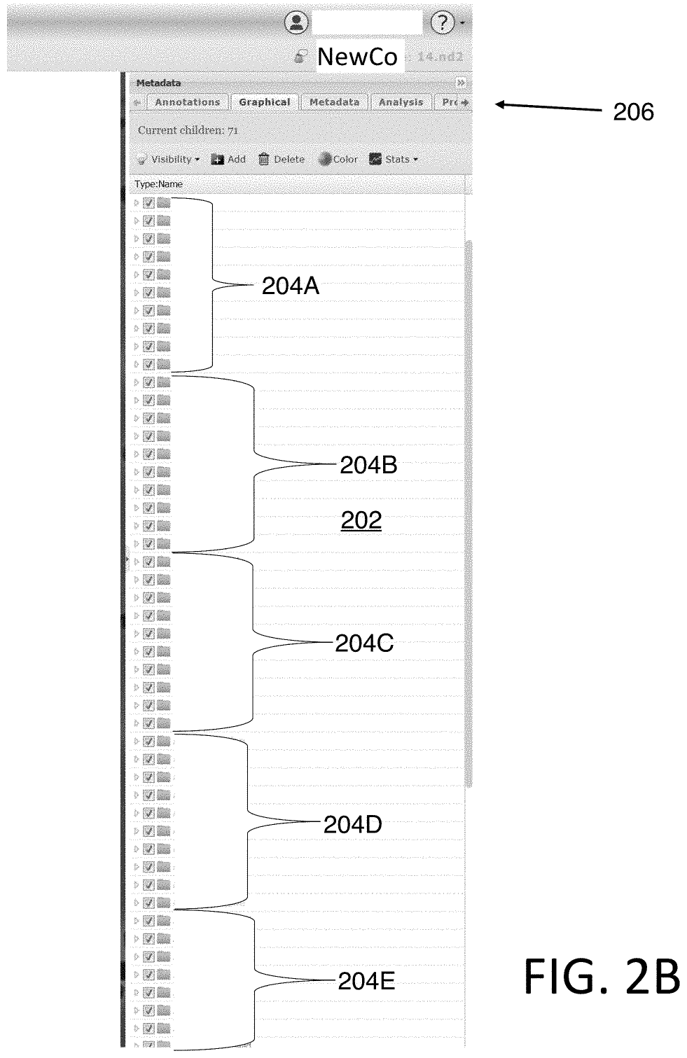

The platform supports all popular microscopy image formats (including channels and time series). The platform has a slide viewer that allows visualization and annotation of images of any size, with multiple channels and of any bit depth. FIG. 2A shows a user interface 200 with an example high-resolution image of a portion of a biological sample in a viewer window 201. The example high-resolution image is a five (5) channel, sixteen (16) data bit-width image taken from a high resolution digital microscope or other high resolution digital imaging device (digital imager). In one embodiment, the digital imaging device is an imaging robot that performs robotic microscopy. Additional robots, such as a fluid handling robot, can be used to process the plates for imaging in order to provide a more automated process.

An important criteria of image acquisition and analysis is the primary acquisition and timely transfer of images for cloud processing. The high resolution images tend to be large and difficult to transfer by email. The majority of users can utilize internet transfer mechanisms to upload images either directly from the acquisition device or from local network-attached-storage (NAS).

Optimally raw image data, experimental metadata including cell-line, and per well data such as viral dilutions and replicates are needed for an accurate analysis. Ideally, this functionality would be integrated into the acquisition pipeline through close integration into the software controlling the imaging, but these devices may have incompatible or prohibitive security, operational or hardware requirements.

For instances where there can be incompatibilities or other issues with integrated acquisition of data, custom software can be used to interrogate the client software for the needed data. Toolkits and services can be deployed locally to automatically and securely transfer images and metadata as acquired. In some cases, if direct access to the acquisition machine and its raw image data is not possible, raw image data can be manually or automatically imported or exported. A toolkit can be structured so that collection of raw image data can be imported with multiple strategies: from application database, from filesystem export, etc. Experimental meta-data is usually not reported by imaging devices unless they are part of a LIMS (laboratory information management system). The meta data is acquired with the raw image data.

Experimental layouts are represented on a grid using a spreadsheet. Spreadsheet templates for experimental layouts can be provided to clients to facilitate the uniform importation of data. Parsers and web user interface (UI) components can also be used on user-provided spreadsheets. Alternatively, images and metadata can be transferred directly through the web browser via a proprietary web portal. In the event of limited or no connectivity due to poor infrastructure, regulatory, or security requirements, local storage and processing can be made available for large-scale users.

Referring now to FIG. 1B, a high-level block diagram of a biological cell analysis system 100 is shown that utilizes machine learning to detect early-stage cellular morphological changes. The biological cell analysis system 100 includes an image processing system 102, a machine learning (AI) model 104 that can be either trained or used for classification/analysis, and a user interface 106. The machine learning (AI) model 104 can be trained for use with one or more machine learning algorithms (including image processing algorithms) and then used with the one or more machine learning algorithms for classification/analysis of objects within images of biological samples (infected, uninfected, treated or untreated), including biological cells and their sub-cellular structure.

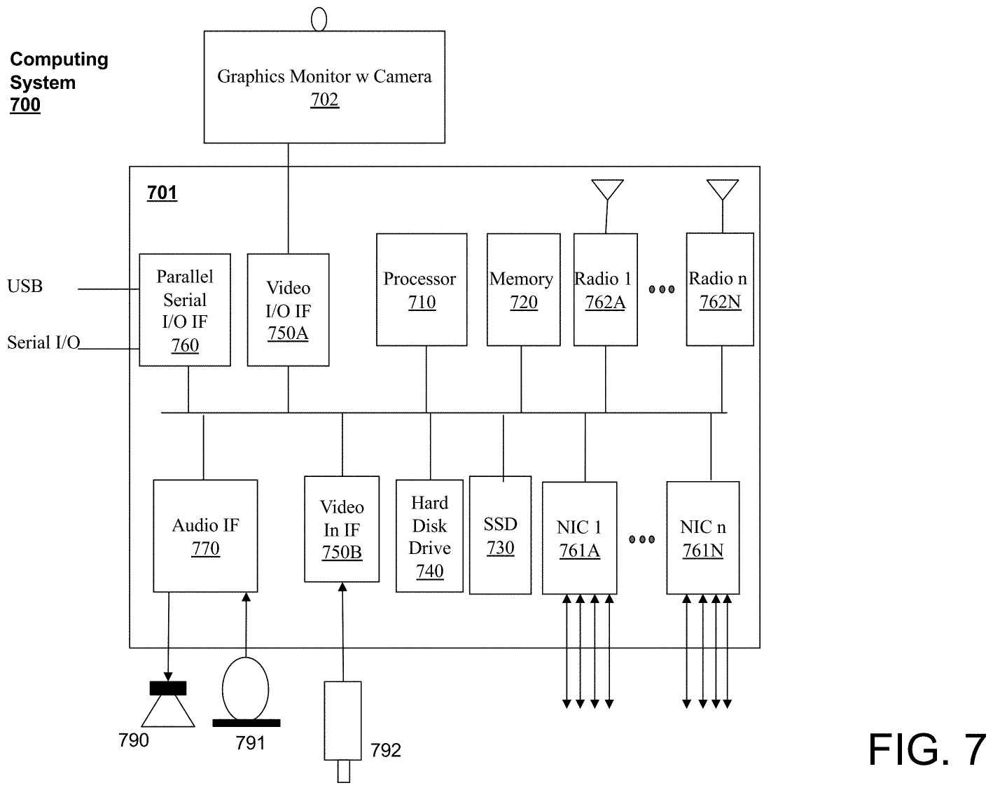

The image processing system 102 can read digital images of cells stored in a storage device 124 and associated metadata from a database 101. The raw image data and associated metadata can be respectively transferred from a user's storage device/database via the internet into the storage device 124 and the database 101 for cloud image processing. In one embodiment the database 101 is stored in the storage device 124 with the images while in another embodiment, the database 101 is stored in another storage device (e.g., memory 720, SSD 730, Disk Drive 740 shown in FIG. 7) separate from the storage device 124 storing the raw image data. In other cases, the database 101 and the storage device 124 can be local and local processing by one or more local processors with local software can be utilized for clients having sufficient hardware and volume of stored assays.

Once the image data and experimental metadata are available in the database 101, the image processing system 102 can be used to process raw image data. The raw image data can be processed to obtain specific image information (e.g., resolution, size, background filtering, etc.) The image processing system 102 can generate feature vectors from the captured images of the biological cells. The feature vectors can be used to train the machine learning (AI) model 104.

The machine learning (AI) model 104 can be trained to provide useful and accurate information about the biological cells and other objects within a biological sample. In one embodiment, the machine learning (AI) model 104 is used with one or more classifier algorithms to classify biological cells and to detect morphological changes of the classified biological cells over time.

Once the image data is processed by the image processing system 102, the image data can be used to train an AI model 104 in a training mode 105A. The AI model 104 can be validated with additional image data that is excluded from the training process. If the AI model 104 has been previously trained (pretrained), it can be used in an analytical mode 105B to analyze biological cell assays. In the analytical mode 105B, images of biological cell assays can be analyzed to recognize and classify biological cells. Counts of the number of classified cells can be made within the given area of the images. From the counts, results and accuracy of predicted concentrations can be generated.

A user can interact with the system 100 through the user interface 106. The user interface 106 can be used to build one or more AI models for a new sample of biological cells. The user interface 106 can be used to seed the recognition/classification of one or more objects in the new sample. The user interface 106 can receive the report generated by the use of the AI model 104 and algorithms in analyzing the one or more images of a biological sample. The report and analytical results can be viewed in various windows generated by the user interface 106 on a display device.

In a diagnostic setting, the report may include diagnosis information of a patient associated with the biological sample, or the sample can be from environmental monitoring. The report can include percentages of likelihood of the presence of one or more infectious viruses, which can inform possible treatment information for the patient.

FIG. 2A illustrates a user interface 200 with a display window 201 displaying a brightfield image of biological cells in the brightfield channel. FIG. 2B illustrates the right panel 202 of annotations of different tags or labels 221-224 having different colored circles on a few objects recognized by a user within the image 201 shown in FIG. 2A. The objects tagged or labeled in the image can be recognized by machine learning with annotations (type of object, location) added as part of the metadata 206 of an image. The tags or labels objects in the image can be colored differently, such as by different colored circles, and can be overlaid onto the objects (e.g., cells, debris) such as shown in the image of FIG. 2A. The different colors can be used to emphasize the annotation and provide information about the tagged object, such as a cell or a subcellular component (e.g., nucleus, lysosomes, peroxisomes, mitochondria, endoplasmic reticulum, golgi apparatus) and its state (live, dead, infected, uninfected).

As shown in FIG. 2A, the annotations associated with the tags or labels 221-224 can include Debris 204A in a first color, CellX_live 204B in a second color, CellX_dead 204C in a third color, CellY_live 204D in a fourth color, and CellY_dead 204E in a fifth color, for example. The annotations shown in FIG. 2B can be colored to match that of the colored circles overlaid onto the objects (e.g., cells, debris) shown in the image of FIG. 2A. The tags or labels 221-224 on the objects and associated annotations, are used to train one or more AI models of supervised machine learning algorithms so that the same classes of objects can be recognized throughout the image at different locations.

### Machine Learning Service

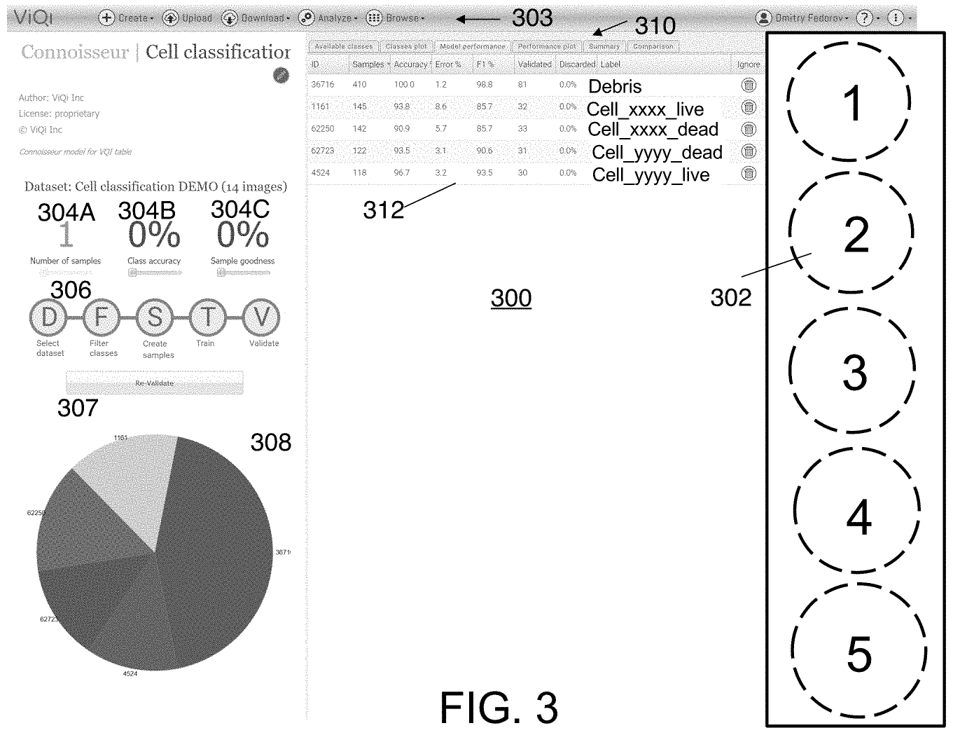

Referring now to FIG. 3, the web-based scalable image analysis platform 10 shown in FIG. 1A, includes a machine learning service as part of the Analysis Services 18 to build AI models and classify cells and other objects. FIG. 3 illustrates a deep learning model builder 300 as part of the platform. An example imaging AI cell classification model shown in FIG. 3 is used for classifying live/dead cells in a sample of cells. The model builder 300 illustrates a sample 302 (sample two of one through five) of a training dataset that is shown in the right side of the frame. A row of control buttons 303 at the top of each page, including Create, Upload, Download, Analyze, and Browse, can be use for creation, uploading, downloading, analyzing, and browsing.

A slider and display 304A can be used to filter out classes based on minimum number of samples per class. A slider and display 304B can be used to select a minimum accuracy required for a class to be used in classification. A slider and display 304C can be used to select a goodness threshold under which classification results for individual samples will be discarded during classification. A progress bar 306 of the steps in building the AI model is provided, including selecting the dataset, the filter classes, creating samples, training of AI model, and validating the AI model. The user interface guides the user through each of these steps of the process, shown by the progress bar 306. If something changes in one or more of the settings, a revalidate button 307 is provided to re-validate a previously trained and validated AI model. The builder includes a pie chart 308 that visibly shows, with different colors, the size of the different object classes found in the training dataset.

The builder 300 further shows a plurality of tabs 310 that can be selected to show information about the AI builder including available classes of object that can be recognized, a plot of the classes of objects used during model building, model performance table (shown), a performance plot of objects, a summary table, and a comparison table. The selected tab (model performance) shows a model performance table 312 of the object classes recognized by the AI model for the selected training dataset. It is information used during training of the AI model. For example, in a row of the table 312 for the class of Cell_xxxx_live cells having ID 1161, the AI model and classifier included 145 cells from the training dataset, resulting in a 93.8 percent accuracy, an 8.6 percent error rate, an 85.7 percent F1% rate (a metric quantifying the prediction accuracy), with 32 cells from the training dataset used for validation, and 0.0 percent of the cells discarded during training.

The machine learning service allows a user to build machine learning models on large-scale training data collected from images and metadata with a variety of features and learning frameworks. The generated models can be used to predict properties over large image collections and visualize the results as overlays for easy digestion by scientific users.

The machine learning service can train an AI model that can distinguish brightfield images of virus-infected cells from uninfected cells at a subcellular level. Training AI models to find initial infection events in brightfield imaged cells, enables the creation of a fully automatable virus infection assay with same-day turnaround.

Typically, early infection events are not visible to human observers. Membrane-bound structures associated with viral infection can be seen by electron microscopy and they are of sufficient size to have a readout in visible light, but they have not been observed manually with light microscopes. In contrast, AI models have been trained to observe many phenotypic effects in cells using brightfield microscopy that are invisible to human observers. It has been demonstrated that AI models can be trained to detect virus infections in assays as early as four hours after infection using different viruses in different labs from digital images captured by different microscopes.

Traditional infection assays require days for the cells to go through multiple rounds of infection so that cell death resulting from infection can be easily detected. Alternatives that may speed processing somewhat, rely on fluorescently labeled antibodies to viral proteins. However, fluorescent labeling requires many processing steps and expensive reagents, or the development of viruses carrying genes for fluorescent proteins, which are difficult to develop and can affect viability of the virus. Accordingly, it is desirable to detect virus infections in assays without fluorescently labeled antibodies or viruses carrying genes for fluorescent proteins.

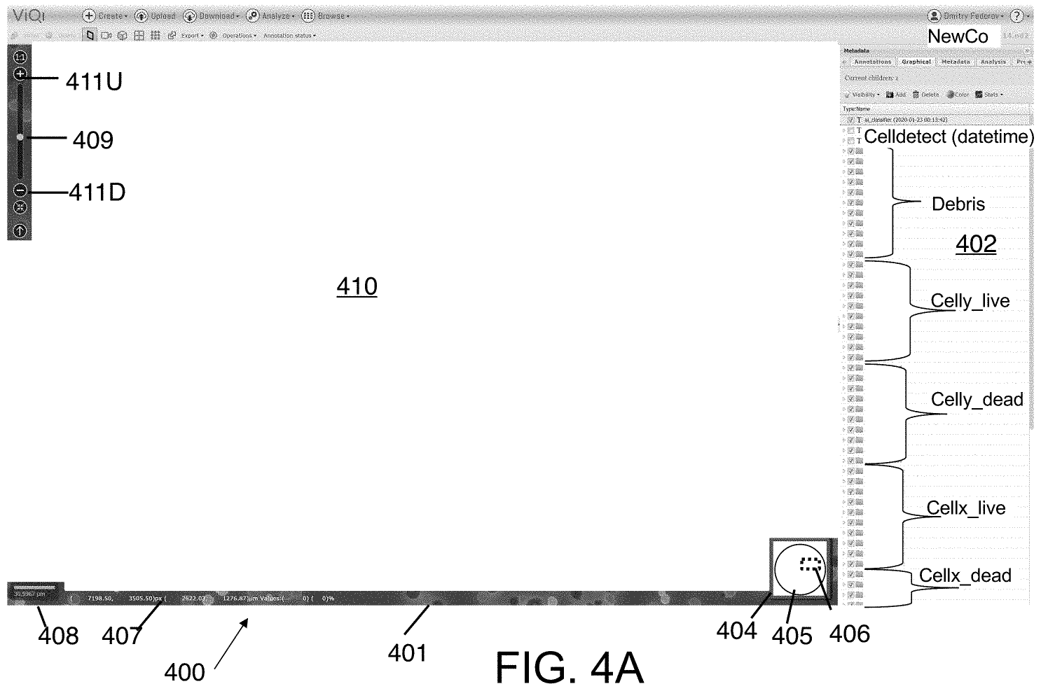

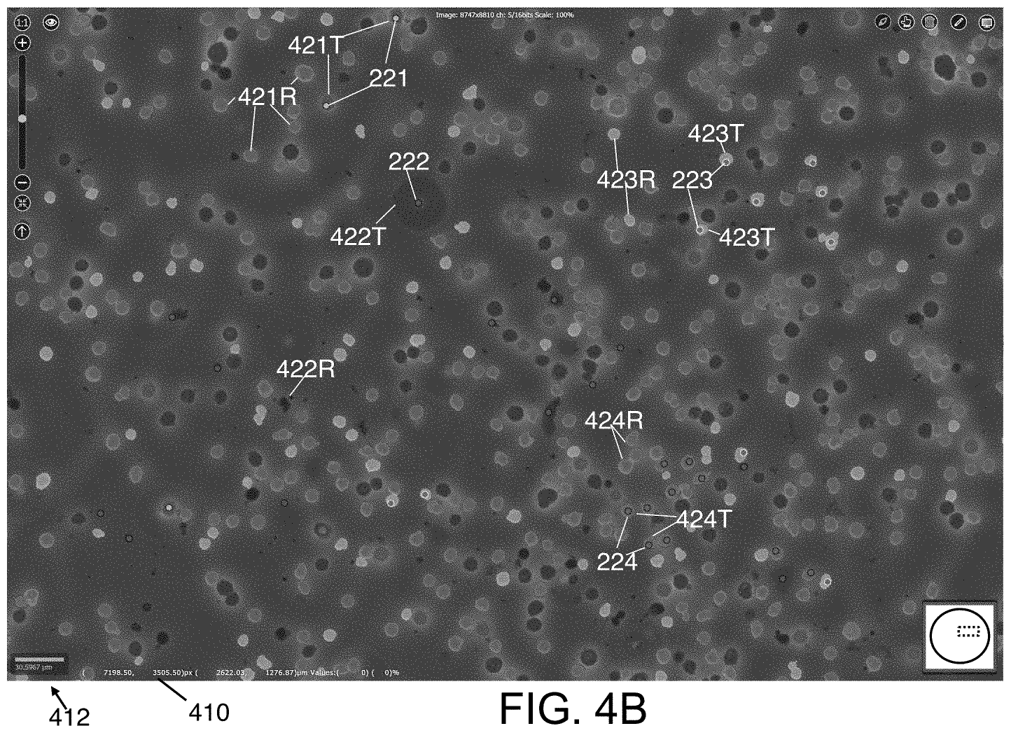

FIGS. 4A-4B show a user interface window 400 with an example resultant output of a machine learning classification with classified (e.g., live/dead) cells and other objects recognized in brightfield images and color coded using an object outline overlay. In FIG. 2A, tags or labels 221-224 were added on top of objects and associated annotations were created. In FIG. 4B, these tags and labels 221-224 are used to train one or more AI models of supervised machine learning algorithms to train on the training objects 421T-424T. With these trained AI models, the supervised machine learning algorithms can recognize the same classes of objects as recognized objects 421R-424R throughout the image at different locations during analysis after training. Besides tags or labels added by a user, AI models can be trained using fluorescent tags (fluorescent protein genes, fluorescently labeled antibodies, etc.) to mark cells of interest.

Multi-channel imaging with a fluorescence channel and a brightfield channel can be used to train AI models. Viruses containing fluorescent protein genes, or staining the cells with fluorescently-labeled antibodies can be used to detect viral proteins and distinguish infected from uninfected cells in the fluorescence channel. Then, the AI model can be trained by looking exclusively at the brightfield channel at cells identified as infected vs. uninfected by using the coincident infection signal in the fluorescent channel. A trained AI model can be validated by having it make predictions on the cells using the brightfield channel alone, and using the coincidence of the AI's infected predictions with the signal in the fluorescence channel to determine AI model accuracy. After training and validation, the AI model can then be used solely on the brightfield channel, without the fluorescent channel, during analysis of other brightfield images of infected cells.

In FIGS. 4A-4B, the viewer overlays the classification results on top of the image as color-coded masked regions. For example, the color red can be overlaid onto certain cells to indicate the dead cells to the user. The color yellow, for example, can be overlaid onto certain cells to indicate live cells. Other colors can be used instead and other cell states can be indicated by more colors as well. The visualizer (platform slide viewer) allows for smooth navigation of large images, even with millions of such regions.

As shown in FIG. 4A, the user interface window 400 includes a viewer window 401 and a side bar 402. In the viewer window 401, a magnified portion 410 of the sample is shown. The view window 401 includes an overview window 404 of the sample in a well 405. The portion 410 of the sample shown in the viewer window 401, is indicated in the overview 404 by a rectangle 406 in the well 405. Magnification information 407 is overlaid onto the portion 410 in the viewer window 401. A scale 408 is overlaid onto the portion 410 displayed in the viewer window 401. Magnification adjustment controls 409, 411U, 411D are overlaid on the portion 410 to allow adjustment of the portion 410 of the sample displayed in the viewer window 401. A slider 409 can be selected by a user input device and adjusted up or down to magnify or demagnify the portion 410 of the sample displayed. An up button 411U can be pressed by the user input device to zoom in on the sample portion displayed in the viewer window 401. A down button 411U can be pressed by a user input device to zoom out on the sample portion displayed in the viewer window 401.

After an analysis (prediction or classification) is run with the AI model on one or more images of cells, the side bar 402 can indicate the various objects (e.g., infected cells, uninfected cells, subcells) recognized and displayed in the viewer window 401. The side bar can also indicate the AI model that is used to generate the objects and the date and time the analysis is run with the AI model. The side bar can also indicate the user provided metadata that was used to train the AI model.A

The magnified portion 410 of the sample shown by the viewer 401 is illustrated in FIG. 4B. Over that of the viewer window of FIG. 2A, objects are detected, classified and shown by the different colors after an analysis.

In addition to the above-mentioned features, the web based platform has built-in support for sharing and collaborating among scientific teams. Because it is substantially web-based, no complex software installations are required to get started analyzing biological cells. The capabilities of the web-based scalable image platform 10 make it easy to use the system for the early detection of changes in cellular structure.

The disclosed embodiments train imaging artificial intelligence models (AIs) to detect individual, unlabeled cells infected with virus. This is innovative because machine learning directly on brightfield images without stains or other additional preparations has never been accomplished for virus infectivity assays. This allows such assays to be conducted on platforms compatible with HCA automation workflows, without the need of costly and time-consuming additional staining, sample preparation or development of transgenic viruses carrying fluorescent protein genes. Furthermore, the insights gained during the development of the models are useful for other cell-based assays.

In addition to the innovative machine learning aspect, the developed models are shareable worldwide using a web-based platform. This is innovative as model sharing in the past meant setting up complex software systems to read such models and then performing classifications based on them. By using a web-based platform, sharing of models merely requires a web-browser. Efficient sharing of models among scientific teams is crucial for the rapid development of urgently needed vaccines or anti-virals.

### Training and Classification Workflow

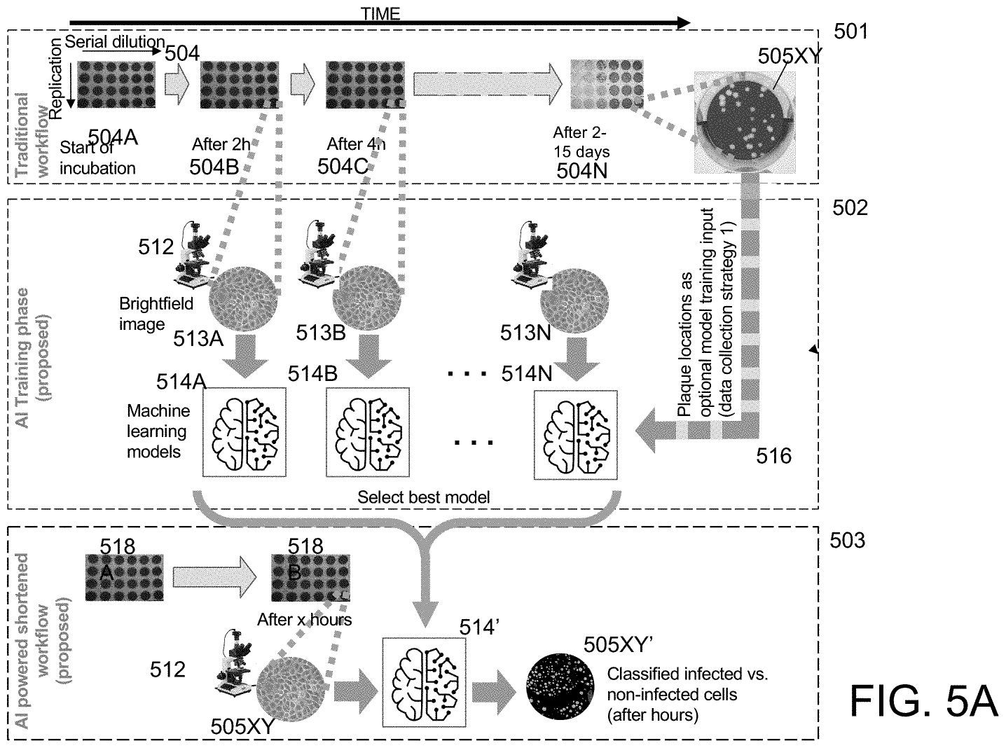

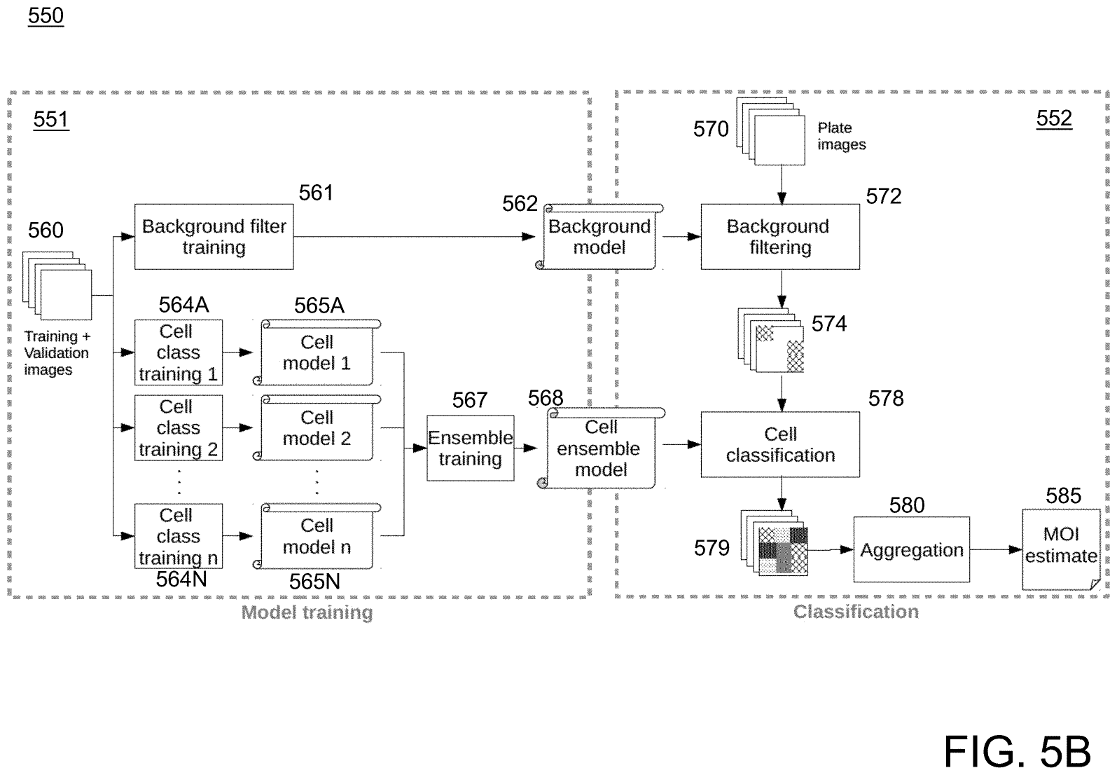

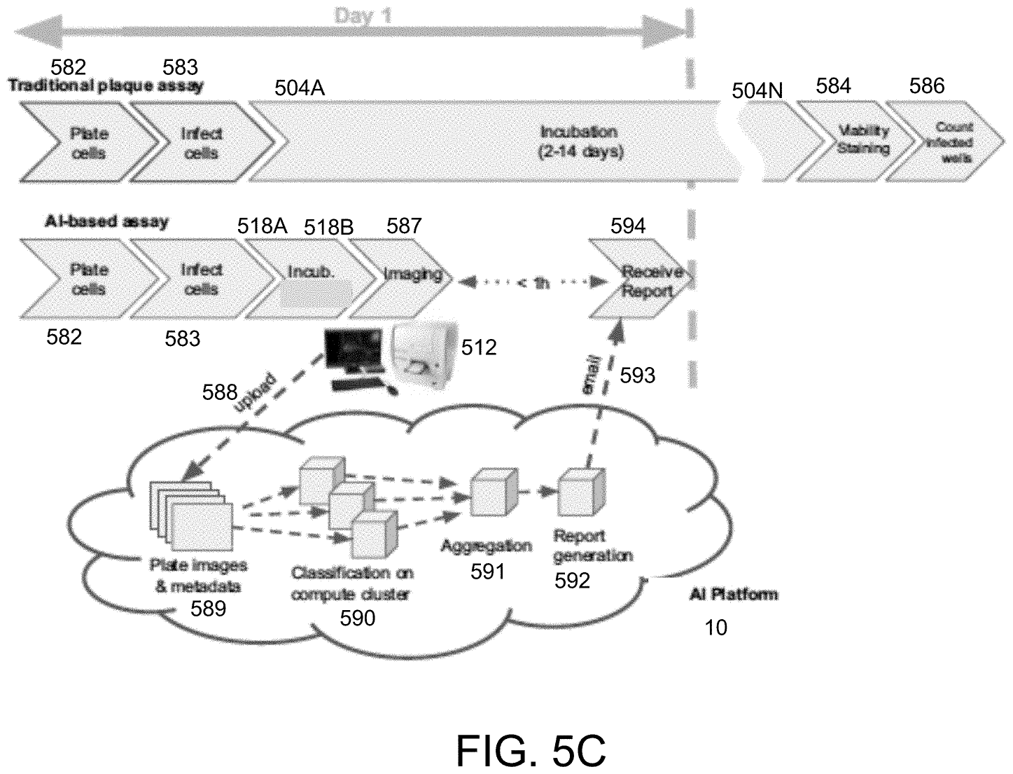

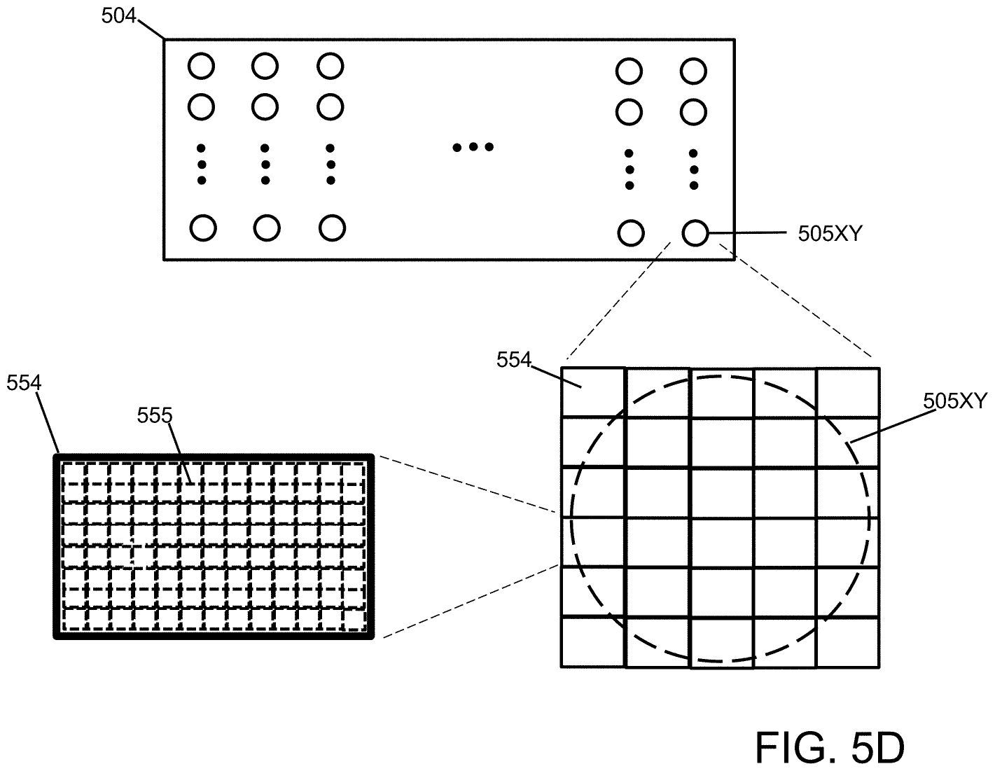

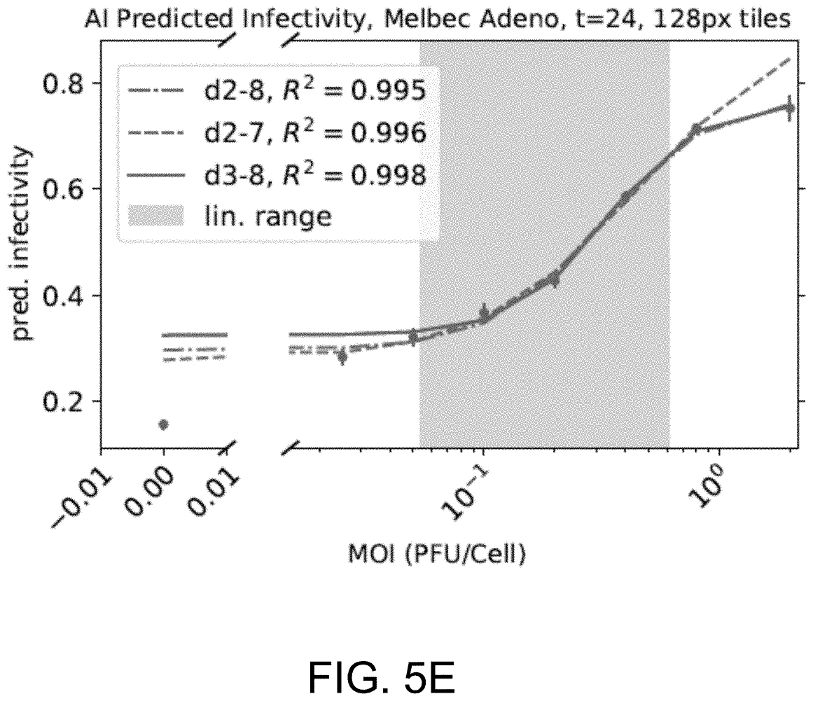

One main objective of the embodiments is to reduce the time to obtain plaque assay results by an order of magnitude (e.g., from days to hours) via the use of machine learning. Referring now to FIG. 5A, panels 501-503 are shown to discuss different assay workflows. Panel 501 (top panel) of FIG. 5A shows a traditional assay workflow. Panels 502-503 (bottom two panels) of FIG. 5A show an artificial intelligence (AI) enhanced workflow with a training workflow and a classification workflow.

Training of AI models can use images of wells of cells infected with a virus at a multiplicity of infection (MOI) of two or greater, and images of wells of cells that are mock-infected. Subsets of these images can serve as the positive and negative classes for training the AI model. These images are captured at different times after infection ranging from a few minutes (zero hours) to forty-eight hours after infection. Images of wells with cells infected at MOIs ranging from 0.8-0.025 can be used for the infectivity assay validation dataset. The multiplicity of infection (MOI) can be defined as the ratio of the number of live infectious virus particles per cell.

In FIG. 5A, the traditional assay workflow shown in panel 501 can be contrasted against the artificial intelligence (AI) enhanced assay workflow shown in panels 502-503.

Both traditional and AI workflows start with an array of a plurality of incubated wells 505XY containing infected cells. The plurality of wells 505XY can be in a tray (plate) 504 and organized by column X and row Y. A serial dilution of infected cells can be placed in the wells of each column along a row. The serial dilution of infected cells in the wells can be repeated in one or more rows so that a plurality of rows of serial diluted cells are present in the wells of the tray at the start of incubation 504A. After two hours of incubation of the tray 504B, a plurality of digital images 513A of each well in the tray 504 is captured by an image capture device 512, such as a microscope or automated imager. After four hours of incubation of the tray 504C, a plurality of digital images 513B of each well in the tray 504 is captured by the image capture device 512. After additional incubation periods (hours, days) of the infected cells in the tray 504, a plurality of images can be captured at each incubation period by the image capture device 512. After a plurality of days 504N (e.g., two to fifteen days), a final set of a plurality of images 513N can be captured to the plurality of wells 505XY at the final incubation period by the image capture device 512. The plurality of images 513A-513N of the wells in the tray at each period can be used to train machine learning models 514A-514N. After verification, one or more of the machine learning models 514A-514N can be selected as the analysis AI models 514′ and used to classify cells in one or more wells of a tray (plate) 504 that are infected with an unknown concentration of infectious agent.

With the traditional assay workflow shown in panel 501, results are provided in a readout in the form of “holes” in the culture where plaques have formed after two to fifteen (2-15) days. Results taking days to generate can be a significant bottleneck, especially when time is of the essence, such as in COVID-19 antivirus/vaccine development for example.

Referring still to panel 501 of FIG. 5A, the standard viral plaque assay begins with serial dilutions of the virus sample to achieve an infection rate of five to one hundred (5-100) plaque forming units (PFUs) per plate or well of a multi-well plate. The diluted sample is applied to a nearly confluent lawn of susceptible cells, and the cells are incubated for two to fourteen (2-14) days to allow the infection to proceed through several rounds of re-infection. The plates can be stained with a viable stain (usually crystal violet) to expose the patches of dead cells that constitute each plaque. Each plaque represents a single infection event followed by multiple rounds of localized virus production and re-infection. To keep the infection localized, the virus produced in the first round of infection is kept from diffusing throughout the well by overlaying the cells with an agarose gel.

The disclosed embodiments use machine learning techniques to reduce the timeline to receive assay results. In panel 502 of FIG. 5A, during a training phase, brightfield microscopy images are taken at one or more time points. These brightfield images contain thousands to millions of tissue cells and are used to train machine learning models (optionally including the final assay result as training data). It is expected that the earliest models (e.g., model 514A) will have less predictive power because the cells have not yet transformed enough from the viral infection. As part of this training phase, the earliest model is selected that provides prediction quality similar to the traditional assay.

Panel 503 of FIG. 5A illustrates an AI powered prediction phase. During the prediction phase, the selected model is then used to classify cells at a time point corresponding to the selected model. The result is a per-well prediction of the degree of infection (predicted infectivity), being made within a much shorter time period (such as within hours, instead of days).

Specifically, one disclosed embodiment can perform the following functional steps:
Collect a time-course of viral infection using high-resolution brightfield microscopy on unlabeled cells. The disclosed embodiment can collect images of several hundred to a few thousand cells at each timepoint that are unambiguously positive for infection and the same number of images of uninfected cells under similar conditions.
Collate annotated image data, train artificial intelligence models (AIs) at various timepoints, and assess performance compared to standard plaque assays. The disclosed embodiment can use artificial intelligence models (AIs) to measure live virus titers with at least the same accuracy as the standard plaque assay, but in a much shorter time.
Extend a web based platform with problem specific machine learning mechanisms and visualizations. One or more disclosed embodiments includes a platform to automate plaque assays, use brightfield images without viable stains or molecular markers, and get an infectious virus titter in hours instead of days. The ability to detect single infected cells and differentiate viral infection with sufficient accuracy from other types of cellular distress, dramatically alters the time and effort typically required for these types of assays, revolutionizing the ways that this type of assay is used in virology.

To train AI machines to discern infected cells from uninfected cells, high-resolution images of cells undergoing infection taken in a brightfield (e.g., phase contrast) channel are collected. Cells in the early stages of viral infection do not have morphological changes that can be discerned manually. Thus, the data collection strategy accommodates for training AI machines on infection-positive cells using images that have no visible differences to control uninfected cells. For contingency purposes, two alternative embodiments were performed based on the capabilities of data collection partners.

FIG. 5B is a block diagram of the artificial intelligence (AI) workflow (process) 550, including a training workflow (process) 551 and a classification (analytical) workflow (process) 552, for predicting viral infectivity of samples of biological cells under test. After images of the biological cell samples are captured, the training workflow 551 generates a background artificial intelligence model 562 and a cell ensemble artificial intelligence model 568 that can be used by the classification workflow 552 to estimate or predict viral infectivity in a biological cell sample under test.

The ensemble model is a machine learning technique that combines several base AI models together in order to produce one optimal predictive AI model. The goal is to find a single model that will best predict the desired outcome. Rather than making one model, ensemble models take a plurality of models into account, and average those models to produce one final model.

The training and validation images 560 are preferably processed from raw image data to form rectangular image tiles 555 of M by N pixels. The M pixels by N pixels of a tile can number in the range inclusively between thirty-two pixels by thirty-two pixels and the entire pixel width and pixel height of the one or more captured images. For example, a tile can be 128 pixels by 128 pixels or 256 pixels by 256 pixels. FIG. 5D illustrates rectangular or square tiles 555 for the image 554 of infected cells in a well 505XY. In an alternative to tiles, cells can be isolated in an image area referred to as an isolated cell image. Although tiles are preferable, the disclosed principles are applicable to both isolated cell images and tile images. The AIs can be trained to recognize viral infectivity on a cell-by-cell basis or a tile-by-tile basis.

The use of larger tiles may help ensure that the assumption that all tiles from infected wells are infected is correct more often than it is for smaller tiles. Down-sampling the 40× images to 20× resulted in better performance for the classifier despite using 4 times fewer tiles for training (and a same-size tiles in each case). However, training the AIs with larger tiles (or downsampled tiles) has the downside of losing resolution in the final assay. This can be partially alleviated with overlapping tiles when processing the assay, but the ultimate resolution of the assay will still be dependent on tile size.

With images obtained using a 20× objective, a tile of 256×256 pixels (256 px) is slightly larger than a typical cell, while a tile of 128×128 pixels (128 px) is smaller than individual cells. Using larger tiles can result in more accurate predictions, despite the 4-fold lower quantity of tiles in training. There are tradeoffs between tile size, resolution and accuracy that can be balanced, so embodiments can interchangeably use both 128 px tiles as well as 256 px tiles.

Blocks 561 and 562 train AI to prefilter background from the training and validation images 560. Because of plating limitations, some tiles can contain background that will decrease accuracy. To alleviate this problem, a “prefilter” network is used to eliminate tiles that do not have cellular material. Background filter training is performed at block 561 to produce a background model AI at block 562 which can perform a background filter step at block 572 to discard tiles that have no cellular material. An advantage of the “prefilter” network and background filter step 572, it is not necessary for training and validation images 560 to be of individually isolated cells.

The other workflow path of process 551, involves training one or more AIs to detect morphological changes at the cellular level. Typically, training an artificial intelligence model that can recognize whatever you want it to recognize in images, involves exposing the AI to a dataset of images and telling the AI which subset of those images of what you want it to recognize. For example, to distinguish between dogs, cats, and stop signs, the AI would have to be told which of the images in the data set are dogs, cats, and stop signs.

The AI, in the disclosed invention, is being trained to distinguish between infected and uninfected cells in a brightfield image. However, unlike in a typical AI training scenario, no human observer can tell the difference between infected and uninfected cells at this level of magnification and time of infection. Simply put, an AI can distinguish morphological changes in a cell that would elude a human observer even when both are ‘looking” at the same images. Brightfield images, without the use of stains and dyes, contain too much information for a human observer to recognize changes. AIs are better at synthesizing images to recognize the differences. Thus, the AI training has to be established in a specific way to train the AI to distinguish what a human observer cannot.

The method of training one or more artificial intelligence (AI) models to differentiate between infected cells and uninfected cells begins by exposing the AI to a dataset of images of stain free cells infected with a known virus stock and images of stain free uninfected cells. As mentioned above, the AIs have to be trained in a specific way that is different from traditional transfer learning methods where different types of images are already known. In the above example cats, dogs, and stop signs are already known to the observer, thus a traditionally trained AI can simply be told which images are dogs, cats, and stop signs. Human observers, however, cannot distinguish between infected and uninfected cells at the magnification and timepoint after infection utilized in embodiments of the invention.

Thus, to train the AI used in embodiments of the invention, the training dataset consists of images of wells infected at a known high multiplicity of infection (MOI) ratio such that all or nearly all cells in the infected cells are infected with virus and none of the cells in the mock infected sample are infected. The dataset used in one embodiment of the invention consisted of images of wells infected at a multiplicity of infection (MOI) of 2 for the infected cells used in training and 0 MOI for the mock infected samples.

The training images are periodically captured at predetermined times over an incubation period ranging from zero until such time that cytopathic effects (CPEs) can be manually observed after initial infection. CPEs are structural changes in the host cells caused by viral infection, e.g., rounding of the cell. If the virus is known to lyse nearly all of the infected cells in 12 hours, than the training images would not be captured beyond 12 hours. In embodiments of the invention, the AI models were trained at 1, 4, 8, and 12 hours timepoints after initial infection.

The preferred method of training an AI model for viral assays, is to train level-1, individual AIs independently in parallel, with the results brought together to train a level-2 ensemble AI. The ensemble AI model 568 uses a two-layer stacking approach to training. A plurality (e.g., 12) level-1 cell AI models 565A-565N are each trained during cell class training 564A-564N using configuration files specifying starting models and their training parameters. The resultant trained cell AI models 565A-565N are used to generate predictions on the images of biological cells withheld from the training images. These predictions of infectivity are used during ensemble training to train the level-2 classifiers, the cell ensemble model 568. The level-2 classifiers are in turn used to make predictions of infectivity on the separate validation images of the biological cells.

In an example of the above process 551, three Convoluted Neural Networks (CNNs), and a total of 4 parameter settings for each of the three networks were trained on images of infected cells at step 564A-564C. The purpose was to demonstrate that an ensemble can outperform the best constituent AI, which it does. In this example, only three CNN models were trained, however there are more CNN models that could be used, and more parameters, potentially doubling or tripling the number of constituent AIs in the ensemble.

The morphological changes between infected and uninfected cells at the early stages of infection are subtle. They are not observable by a human observer. These morphological changes can differ between cell lines. Thus, an AI model trained on one cell line may not be able to differentiate between infected and uninfected cells in a different cell line without further training.

While the AI model and the associated algorithms may not perform an analysis of viral assay as accurately on a different cell line, this statement should not be interpreted in the absolute. For closely related cell lines, the morphological changes may be similar enough to get an accurate viral assay. Although an AI model trained for a specific cell line may not accurately perform a viral assay on another cell line, principles of the disclosed embodiments are still applicable to all cell lines. In any case, the method of training an AI model for an infectivity assay remains the same across all cell lines and all viruses. Thus, the disclosed embodiments can assay all cell lines and all viruses with trained AI models.

| | RNA | DNA |
|---|---|---|
| Enveloped | HIV, Influenza, 229E | Vaccinia, MVA |
| Non-enveloped | Polio | Adeno variant, Adeno |

Disclosed embodiments of the invention have successfully assayed enveloped and non-enveloped RNA and DNA viruses. The virus stock used in embodiments of the invention contains virus of the group of DNA (coated or uncoated) and RNA (coated or uncoated). The table above lists the various types of viruses accurately classified using the disclosed AI assisted assay system and method of the invention.

An alternative approach to machine learning involves feature-based classification using CHARM features (a large feature set used successfully in many cell-based AI applications, constituting more than 4,000 numerical image features). These features can be combined with the automated feature-classifier trainer in the final ensemble stage. The trainer can automatically select a pipeline of feature normalization, scoring, selection and classification algorithms from scikit-learn, and automatically optimizes their parameters. Several variants of this technique can be used to supplement the CNNs.

It is expected that the types of errors made by feature-based classifiers would be quite different than those made by the CNNs, thus potentially boosting the overall accuracy. We will also investigate incorporating existing AutoML packages such as Auto-sklearn and AutoKeras for automating specific level-1 and level-2 classifiers in addition to the previously mentioned fully automated feature classifiers.

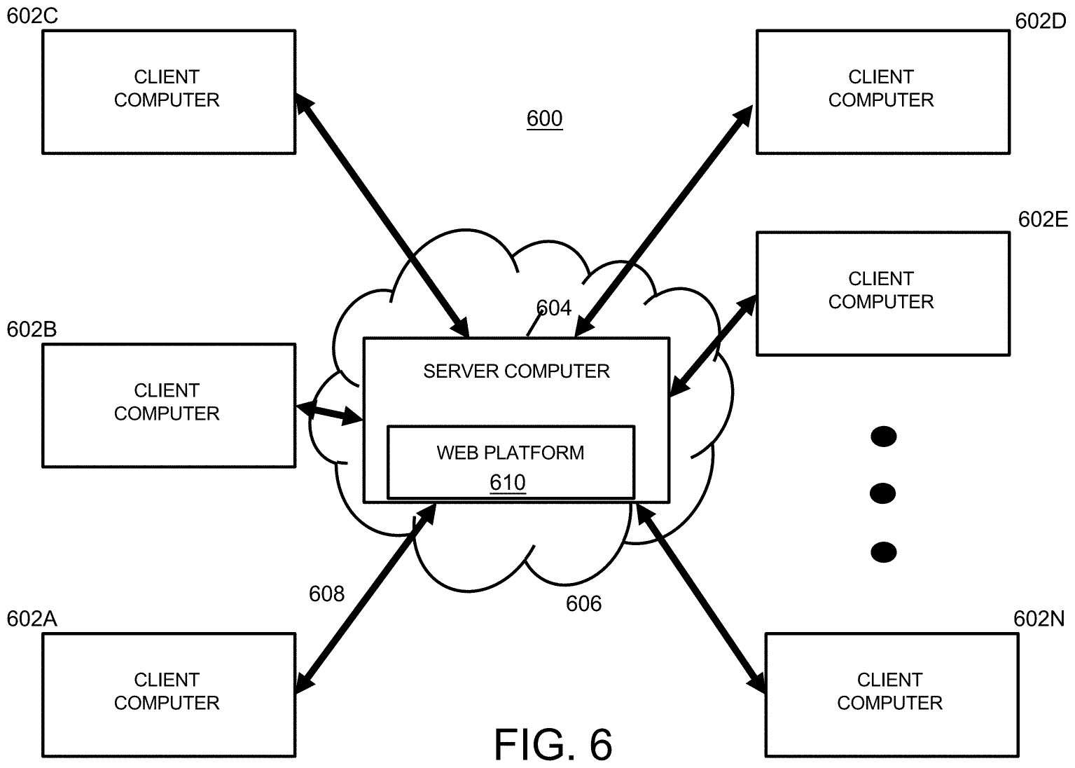

In earlier embodiments of the invention, the training the AIs can be executed manually via a number of scripts. For later embodiments of the invention, each stage of the training/classification process is performed as an analysis module in a computer server system cloud platform (e.g., see FIG. 6), and automates the overall workflow composed of constituent software modules on a compute cluster. This ensures that the data flow of intermediate results of the training/classification workflow are fed correctly into subsequent analyses in an automatic manner, rather than managing data flow manually.

The disclosed embodiments also provide for storage of trained models as outputs of AI training modules and allows them to serve as inputs in subsequent prediction steps. This ensures that the entire dataflow is specified as part of the workflow, which not only aids in organizing various AI models trained using various datasets and parameter sets, but prevents inadvertent mistakes that are possible when these flows consist of potentially thousands of manually handled individual files.

In an example of the process described above, an ensemble model based on three different pretrained network architectures, two learning rates and two dropout rates for a total of 12 trained models for each tile size, were trained with 12 models for 128 px (128×128 pixels) tiles and 12 for 256 px (256×256 pixels) tiles. An automated feature classifier trainer tries several different feature selection and scoring techniques coupled to several different classifiers, tries a range of appropriate parameters for these algorithms and selects the best-performing model and corresponding parameter set. The ensemble model resulted in an accuracy of 81% for 128 px tiles, compared to an average of 70% and max of 77% for the individual constituent models. For 256px tiles, the ensemble network achieved an accuracy of 88%, compared to an average of 82% and max of 86% for the individual constituent models. Varying the parameters ensured the constituent AI models are trained as differently as reasonably possible so that their individual results are as different as possible.

As can be seen from the results, the underlying or constituent models can be decently accurate in their accuracy. However, the ensemble models were significantly more accurate. That being said, it should be clear to any practitioner in the art that the scope of the disclosed embodiment is not limited to either constituents or ensemble models but covers both.

Once an ensemble AI model is trained, the ensemble AI model can be validated. Validation compares the AI model prediction of a known virus' infectivity against the same known virus' infectivity determined by a traditional plaque assay. Although the AI models are trained in the context of a high-MOI training set, it is important to validate these models in the context of a real assay where the MOIs are <1.0. The predictions of the AI models are compared to the results of traditional assays of cells with MOIs in a series of viral dilutions of a stock with known titer. For 256 px tiles, the ensemble AI model can achieve an accuracy of eighty percent or more.

With the AI models (background model 562, and cell ensemble model 568) trained, they can be used in a classification process of infected biological cells captured in plate images 570. In process step 552 of the workflow, heretofore unseen images 570 (at least by the AIs) are classified with objects of the samples in the wells being recognized and counted. The images 570 can be from a single infection in a single well of a typical ninety-six well plate. The images are analyzed and predictions are made by the AI model and the associated machine learning algorithms. These plate images 570 are not used in a training process. The plate images 570 are prefiltered at step 572 by the background model 562 to remove background “noise”, such as any non-cellular material for example, and generate prefiltered images 574. Then, the prefiltered images 574 are analyzed by the ensemble model 568 during the cell (object) classification step 578. This can be performed in parallel on tiles of images. The analyzed images are assigned a score for each tile in the image at process step 579. During an aggregation step 580, the scores are aggregated together or averaged across all the tiles in the image and all the images in a well for a given dilution within the linear range of MOI determined by the validation step. The aggregated score is then used to predict an MOI estimate at block 585.

The analysis or classification process step 552 can be used for viral assays such as an inactivation assay. The AIs are trained and validated on a known virus. Cells infected with the known virus can be subjected to an inactivation agent and then analyzed in process step 552. A researcher can determine from the MOI estimate 585 how effective was the inactivation agent against the known virus. Likewise, embodiments of the invention can be used for oncolytic targeting, clearance assays, diagnostic assays, and creating biocides, vaccines and antivirals.

### Ai Based Vs Traditional Assay

Referring now to FIG. 5C, illustrating a workflow diagram contrasting a traditional plaque assay with an AI assisted assay. In a first step 582 of both traditional and AI assisted assays, biological cells are plated into wells of a play or tray. In a second step 583 of both traditional and AI assisted assays, the plated cells are infected with a virus to form infected cells. The choice of cell lines and viruses are not particularly material to the principles of the disclosed embodiments. The scope of the invention is not limited to a particular cell line or viruses with the caveat that the cell line is susceptible to infection by the virus. The principles of the disclosed embodiments can be applied to any virus and any cell line susceptible to infection by that virus.

At the top of FIG. 5C is the timeline 504A-504N for a more traditional infectivity assay termed the plaque assay. The plaque assay is a critical tool for viral isolation and quantitation. The number of infectious viral particles in a sample is measured in ‘plaque-forming units’ or pfu. Titration of a viral suspension is carried out by serial dilutions of a known virus. The purpose of making and testing serial dilutions is to achieve a “countable” number of plaques in the cell monolayer. If too many infectious particles are present, individual cells are likely to be infected with more than one virion or individual plaques will merge to form large areas lacking cells.

A monolayer of a cell line is plated typically in a soft agar overlay on a petri dish containing the appropriate nutrient agar. The viral suspension titer is added to a monolayer of cells susceptible to infection by the virus used. An agarose overlay is applied to the cell monolayer after infecting the monolayer with the virus. The agarose overlay immobilizes viruses and prevents cross-contamination among plaques. Over the timeline 504A-504N, the plated infected cells are then incubated for a period of two to fourteen days.

Individual infected cells release viral phages that infect the surrounding cells of the monolayer of cells. Multiple rounds of infection can occur until individual holes, or plaques, appear in the cell monolayer. Plaques should be visible by day 4 post infection. At a viability staining step 584, the live cells can be stained, providing a dark background for the clear plaques. At a counting infected cell step 586, the number of plaques in each well are counted. This allows for calculation of the number of plaque-forming units in the original suspension.

In contrast to the traditional plaque assay, the overall AI based assay timeline is much shorter, one day or less in comparison with multiple days for the more traditional plaque assay.

In the AI based assay timeline, at a first step 582, a monolayer of cells is plated into wells 505XY of the imaging plate (tray) 504. In second step 584, the monolayer of cells is infected with a viral titer to form infected cells. In a second step 583, the plated cells are infected with a virus to form infected cells. With a plurality of wells of infected cells in the plate or tray, the incubation 518A period 581A-518B can occur. The infected cells need only be incubated for a few hours (e.g., such as between a range of four to forty-eight hours).

After incubation, the infected cells are imaged at step 587 using a plate imager (image capture device, imaging device or digital imager) 512. The disclosed embodiments can use an automated plate imager capable of producing brightfield images with 20× or 40× magnification. A preferred image collection strategy is to work with a user group working with COVID-19 that are performing plaque assays as part of their normal operations. The user group typically has a microscope 512 able to image the whole surface of a well at high resolution (subcellular resolution) using phase-contrast with 40×-60× resolution. These types of microscopes are commonly available in imaging facilities, but they may be less available under biosafety level three (BSL-3) conditions.

Alternative image collection strategies can also be used. The ongoing infection is imaged at several timepoints (e.g., 2, 4, 8, 16, 24, 36, 48 hours) along a timeline 504A-504N post-infection prior to the development of plaques. These several timepoints ensure that cells are captured in the earliest stages of the infection as well as later stages, and potentially the early stages of the subsequent round of infection. The purpose of imaging each entire well is to use the normal plaque readout at the end-point to localize the plaques at plaque locations 516, and then use those same areas 516 of the well in the previous timepoints to obtain images of infected cells prior to any changes being visible. The plaque locations 516 at the end point can be used as another AI model training input as part of a data collection strategy.

At step 588, the plate images 513A-513N and metatdata are then uploaded to a cloud AI platform 10 where the images are processed. The image processing step 589 can include, downsampling, tiling, filtering background, etc. Image processing 589 and classification steps 590 are preferably performed in the cloud-based (web-based) platform 10. However, clients with weak or no internet, security concerns, sufficient hardware and volume of assays, can utilize client-side software to locally process their image data and run AI assisted viral assays. The processed image tiles are given to the trained AI for a classification or analysis step 590. With image tiles, the classification step 590 can be performed in parallel by multiple processors in a computer cluster, such as provided by a server computer (see server computer 604 in FIG. 6). At step 591, the AI scores for the image tiles and the metadata (annotated objects) can be aggregated together. At step 592, a report of the viral infectivity of the cells is generated. At step 593, the report can then be sent back to the client or laboratory, such as by email. At step 594, the report can be reviewed and the user interface with the viewer window can be viewed by the user.

After the imaging step 587, it can take less than an hour to go through the upload process 588, the processes 589-592 of the AI platform 10, and the transmission process 593 with the report. Accordingly, the AI based assay timeline can be completed in one day or less.

The disclosed embodiments have been developed to receive images from existing COVID-19 user groups. It alters the method for existing plaque assay with additional imaging. The additional imaging posed additional challenges beyond the limited availability of high-resolution whole-well imagers in biosafety level 3 (BSL-3) facilities. Phase contrast and other brightfield methods used to develop contrast in transparent samples rely on structured light transmission through the sample and into the objective. The agarose overlay, typically used in a plaque assay, is not normally required to preserve optical properties of structured light, and so its thickness, optical clarity and composition may interfere with the imaging. However, it is possible to image through agarose overlays, as these are used in chemotaxis studies and studying chemical irritants in cell culture models at high resolution. While these imaging challenges were ultimately surmountable, they did impose additional sample handling considerations on the standard plaque assay.

FIG. 5D illustrates a plate or tray 504 with a plurality of X columns and a plurality of Y rows of wells 505XY. A typical tray may have 12 columns and 8 rows for a total of 96 wells. An imaging device can be used to capture a plurality of high resolution digital images 554 over each of the plurality of wells 505XY in the plate or tray. The digital images 554 of each well can include infected biological cells. In other embodiments with other imaging devices, digital images 554 of a single well, a slide, or a petri dish may be captured with infected biological cells. A digital image 554 of a portion of a well 505XY can be further partitioned into a plurality of tiles 555. Each tile 555 can be rectangular or square with dimensions of M by N pixels. The M pixels by N pixels of a tile can number in the range inclusively between thirty-two pixels by thirty-two pixels and the entire pixel width and pixel height of the one or more captured digital images 554. For example, the dimensions of a tile can be 128×128 pixels or 256×256 pixels. The tiles are preferably confluent with no gaps or overlaps.

FIG. 5E illustrates a validation chart with plots to compare infectivity determined with an AI model and machine learning algorithms versus infectivity determined with traditional assay methods. On the X axis is the known virus with the known MOI determined by traditional assay methods, such as by plaque assays or a median tissue culture infectious dose (TCID50) assay. On the Y axis are the predicted infectivity of the known virus as predicted by the trained AI. The linear region (in green) was determined from the intercept of the linear function extending from the inflection point and the upper and lower asymptotes. The linear range of the MOI was determined to lie between 0.05 and 0.60 PFU/Cell for this exemplary validation step. The linear range can be used by the researcher to determine which dilution of the known virus sample can be used to get a good linear response in the actual viral assay. Thus, the validation step determines an effective MOI range of the assay by validating the one or more artificial intelligence (AI) models against a series of dilutions of a known titer of the known virus stock at an incubation time point determined during the training period. An alternative disclosed embodiment for collecting images of early stages of infection is disclosed herein. Multi-well plates of cells are infected with a sample of pre-tittered virus such that the infection rate is 1-3 PFU/cell. This avoids the need to identify individual infected cells or localized infections because it can be assumed that the bulk of the cells in a given well are infected with a small number of virus particles per cell under these conditions. While there are uninfected cells as well as cells infected with multiple virus particles in this format, this does not substantially affect the trainability of the AI models or their performance accuracy.

Using this training strategy with severe acute respiratory syndrome coronavirus 2 (SARS-CoV-2), that can lead to Coron-Virus-Disease-2019 (COVID-19), directly also requires a plate imager in a biosafety level three (BSL-3) facility. However, the advantage is that the imaging is now completely standard. It does not require any additional processing or staining of the cells, or the use of an agarose overlay. Working with a virus that does not require BSL-3 containment allows this assay to be performed at any standard facility or contract clinical research organization (CRO) with a plate imager that is capable of working with viral samples. It may also be possible to perform this assay with SARS-CoV2 in an automated sample handler as it does not require any manual steps. The Center for Disease Control (CDC) has provided some guidance to indicate that automated sample handlers could potentially be operated within biosafety level two (BSL-2) facilities while using biosafety level three (BSL-3) precautions.

The disclosed embodiments are inherently automated. Previous viral assays included many manual processing steps that could potentially introduce human error into the results. For example, overlaying an agarose layer to prevent unwanted viral infection, is typically done manually in standard plaque assays. Overlaying the agarose layer is a complicated process step, requiring precision temperature control. By eliminating the need for an agarose layer, the disclosed embodiments can be end-to-end automated, increasing throughput and decreasing human error.

The increase in interest in COVID-19 coupled with the requirements for biosafety level three (BSL-3) facilities and surge in demand of BSL-3 facilities, it is desirable to have several data collection options as well as contingency plans for COVID-19 research. A website platform for processing image data from various laboratories and research facilities can be used with trained AI models to more quickly evaluate infectively of samples. Software as a service can be introduced to equipment manufacturers, researchers, and academic institutions rather than having a specific partner. A preferred data collection strategy and a contingency plan based on standard assays and imaging is desirable. In either case, a feasibility study of a virus can still be performed at facilities under the more standard biosafety level two (BSL-2) conditions.

Generally, image data is annotated into annotated image data. The annotated image data is then collated into collated annotated image data. The collated annotated image data can be used to train artificial intelligence models (AIs). The artificial intelligence models (AIs) are trained at various timepoints from the collated annotated image data. The performance of the AI models is compared to the results of standard plaque assays to validate one or more of the AI models.

The training data is collated into labeled training sets of infection-positive cells and infection negative cells. All of the cells in a given well infected at a multiplicity of infection (MOI) of approximately one or greater (MOI of >1) are assumed to be infection positive cells (positive). Cells in wells that were mock-infected have a multiplicity of infection (MOI) of zero (MOI of 0) and are assumed to be infection negative cells (negative).

In one embodiment, a training set of infected cells can be collected prior to plaques being visible or detectable with a viability stain. In this case, it is desirable to detect infected cells prior to lysis. Accordingly, candidate cells are identified that will subsequently lyse or be dead in the final timepoint with the viability stain. This requires first aligning all of the timepoints at a cellular level. While the multi-well plate format can keep the timepoints somewhat aligned as the plate is moved from the incubator to the microscope multiple times, this typically is not sufficient to identify the same individual cells in subsequent timepoints without processing. For this purpose, the embodiments can use several cell segmentation algorithms that work with brightfield images. These algorithms and other algorithms may be used with other digital images of infected cells, such as darkfield images, phase contrast images, and differential interference contrast (DIC) images. The centroids of the segmented cells from each timepoint are used in a random sample consensus (RANSAC) algorithm that aligns subsamples of the centroids of two timepoints to each other and produces affine transforms of the alignments.

These affines were used on the regions of interest (ROIs) defined using the visible plaques in the final timepoint to produce ROIs in previous timepoints where the progressing infection is not visible. Other areas outside of these ROIs are used to identify infection-negative cells for training. Training artificial intelligence models (AIs) on each timepoint independently allows us to identify timepoints that contain a sufficient number of infected cells to accurately differentiate them from uninfected cells. Shrinking the ROIs at earlier timepoints can be used to mimic the spread of the infection and can reduce the number of uninfected cells in the ROI to improve the training at earlier timepoints.

In either data collection strategy, the disclosed embodiments use multiple independent replicates, multiple timepoints post-infection, and several infectivity ratios (PFU/well for a first data collection strategy (strategy 1) and a PFU/cell or MOI for a second data collection strategy (strategy 2). AI performance can increase as the infection progresses with later timepoints. However, the assay's practical value is maximized at the earlier timepoints. For this reason, AI performance is evaluated separately at each timepoint. In one embodiment, six to eight (6-8) timepoints, with three to five (3-5) virus dilutions and three to five (3-5) replicates, is sufficient for AI model training. Each timepoint and dilution is used to train a separate AI model (18-40 total), using all but one replicate for training, and the remaining replicate for testing. Round-robin leave-one-out testing multiplies the total number of AI models by a factor of three to five (3-5) and can produce a measure for the variance in AI performance.

### Dual Machine Learning

The disclosed embodiments trained two different AI systems: feature-based and convolutional neural nets (CNNs). It has been shown that feature-based systems can work well with a relatively low number of training samples, and result in artificial intelligence models (AIs) that can discern differences to high accuracy, that are not visible to trained human observers. Similar results have been obtained with CNNs. Due to a much larger parameter space than feature-based artificial intelligence models (AIs), typically CNN artificial intelligence models require more data to train. Data can be reduced to train CNN artificial intelligence models by the use of transfer learning from an unrelated imaging problem, or the use of data augmentation. These techniques have been used successfully on imaging problems in the past, and they are now part of standard toolboxes for training CNN artificial intelligence models. The performance of the various AI models can be compared at different timepoints to determine which provides better results.

### Data Quantity

Based on experience, the use of three hundred (300) example images per class has been sufficient to achieve saturation training of feature-based AI models. There is no shortage of example infected cells in the second collection strategy (strategy 2). In the first collection strategy (strategy 1), ten to one hundred (10-100) plaques are needed in a well to get an accurate PFU count, and each of these can contain a few hundred cells. Samples can be combined from different virus dilutions at each timepoint, provided that the plaques per well are within the acceptable range. The experimental setup can yield up to a hundred plaques or more at each timepoint (and thus several thousand cells). Thus, there is little need for additional imaging other than the viral dilutions and replicates accounted for above.

### Validation

Once candidate AI models are trained and the accuracy level is comparable to a standard plaque assay using the known outcomes of our training set, the AI based assay can be validated in its final intended form to see if the accuracy is maintained. In a first data collection strategy, the new assay can be tested on standard multi-well plates without agarose overlays to see if the AI model training holds across this change in conditions. In a second data collection strategy, a new infection at an MOI much less than 1 is performed to mimic a standard plaque assay. Because the plaques are now single cells, the system is able to tolerate a much wider range of PFUs per well than a standard assay. This can be demonstrated by finding the individual infected cells in a lawn of otherwise healthy cells, and that the infected cell count can substitute for a standard plaque count.

### Platform Extensions

The web based platform was originally based on BisQue. However, web based platform has been rewritten to make significant improvements over BisQue in many areas including: scalable viewers for data of any size, proprietary storage formats supporting millions of identified objects per image, and scalable deployments. Furthermore, several platform enhancements permit better utilization of resources and faster adoption of novel assays in order to speed the generation of results.

The web based platform has been designed to support multiple machine learning frameworks for both comparison and future-proofing. Caffe deep learning framework is supported. Added support for both TensorFlow and PyTorch/Caffe2 is provided in order to compare the effect of the framework training on result quality. In order to train quickly on novel datasets, we utilize transfer learning based on each deep learning framework's preconfigured neural networks and datasets. Given the levelling of use that the web based permits, different frameworks can be quickly compared. Different neural network topologies can be further used as data becomes available.

Along with traditional deep-learning, a large number of pixel-based features are used to correlate early detection of viral morphology. Specialized visualization is added to demonstrate how certain features better correlate with early detection of viral damage to cells. This visualization takes the form of a heatmap of correlated sensitive features. The information gleaned from such heatmaps can be useful to researchers in understanding specifics of cell morphology. The viewer user interfaces can be enhanced to visualize the internal AI model metrics such as prediction confidence.

There are a number of other advantages to the embodiments disclosed. There is a faster turn-around time in obtaining results. The analysis of cell assays can be more automated and performed more safely on biohazardous cells.

### Diagnostic and Inactivity Applications

As disclosed above, AI assisted viral assays can simplify and speed up the process of determining viral infectivity. The principals disclosed can also be applied to specific types of viral assays, such as diagnostic assays or inactivation assays.

Diagnostic assays are infectivity assays where the virus being sampled is an unknown virus. The virus can be a sample taken from a swab of an infected person. The identity of the virus can be suspected but is not definitively known. If the virus sample is suspected to be a known virus type than the infectivity of the virus sample can be classified by AI trained on the known virus type. For a virus sample that is not suspected to be a known virus, a more generalized AI trained on a plurality of viral stocks can be used to classify the unknown viral sample. Similarly, a plurality of AIs can be trained on a plurality of viruses. Each AI model can report out the probabilities that the cells were infected with the one virus to which the AI model was trained for based upon a comparison of the predicted MOI of the unknown sample and MOI of the virus the AI was trained for. Another variant of a diagnostic assay can be performed in conjunction with biopsies to determine where the viral load is in a patient's body by sampling various tissues and fluids from the patient. In this case the virus is known or suspected, and an AI assisted infectivity assay can determine the location where the virus is localized.

Generally, inactivation assays seek to determine the effects of a treatment or procedure on the infectivity of a known virus sample. A treatment or procedure is conducted on the known virus sample or the infected cell which will affect the infectivity of the viral sample. Treatments and procedures can be with antivirals, biocides, gene modification of the infected cell or virus, drugs, inactivating antibodies in patient sera, etc. A treatment can modify the genes of the virus to express genes for gene therapies. A treatment can modify the genes of the virus to affect the virus's targeting of cells for infection (e.g., oncolytic therapies). A treatment can modify the genes of the virus to modulate the virus's infectivity. A treatment can modify the genes of the virus to modulate the immune response in a host patient (e.g., vaccine development and oncolytic immunotherapies). In any case, the MOI of the viral sample is determined before and after the treatment or procedure to quantify the effects on the infectivity of the viral sample. Embodiments of the disclosed invention can determine the MOI of the viral sample using AI to differentiate infected and non-infected cells before and after treatment.

Subsets of inactivation assays include clearance assays, where a pharmaceutical manufacturing process step is likened to the procedure or treatment in question. The inactivation agent is the process step. A known virus is introduced into the pharmaceutical manufacturing process step and the effects of the process on the amount of the virus before and after the process step must be determined.

Determination of viral infectivity without an inactivation step is required when characterizing new preparations of a known virus for experimental or therapeutic use, or when characterizing new strains developed as candidates for use in gene therapy, or for determining the effectiveness of targeting of a virus to specific cells, such as used for oncolytic viral therapy.

Although diagnostic assays and inactivation assays are disclosed embodiments, it is to be understood that the disclosed embodiments are not limited to these two types of assays. Generally, an AI assisted viral assay is a disclosed embodiment and the principles disclosed are applicable to using AI and AI models to quantify viral infectivity in a viral sample for any purpose.

### Computer Network

Referring now to FIG. 6, a block diagram of a client-server computer system 600 is shown. The client-server computer system 600 includes a plurality of client computers 602A-602N in communication with one or more computer servers 604 in a server center (or the cloud) 606 over a computer network 608, such as a wide area network of the internet. The web-based scalable image analysis platform 610 for biological cells can be executed on the one or more computer servers 604 for access by the plurality of client computers 602A-602N.

### Computer System

Referring now to FIG. 7, a block diagram of a computing system 700 is shown that can execute the software instructions for the web-based scalable image analysis platform 610 for biological cells. The computing system 700 can be an instance of the one or more servers executing stored software instructions to perform the functional processes described herein. The computing system 700 can also be an instance of a plurality of instances of the client computers in the wide area network executing stored software instructions to perform the functional processes described herein of a client computer to provide and display a web browser with the various window viewers described herein.

In one embodiment, the computing system 700 can include a computer 701 coupled in communication with a graphics monitor 702 with or without a microphone. The computer 701 can further be coupled to a loudspeaker 790, a microphone 791, and a camera 792 in a service area with audio video devices. In accordance with one embodiment, the computer 701 can include one or more processors 710, memory 720; one or more storage drives (e.g., solid state drive, hard disk drive) 730, 740; a video input/output interface 750A; a video input interface 750B; a parallel/serial input/output data interface 760; a plurality of network interfaces 761A-761N; a plurality of radio transmitter/receivers (transceivers) 762A-762N; and an audio interface 770. The graphics monitor 702 can be coupled in communication with the video input/output interface 750A. The camera 792 can be coupled in communication with the video input interface 750B. The speaker 790 and microphone 791 can be coupled in communication with the audio interface 770. The camera 792 can be used to view one or more audio-visual devices in a service area, such as the monitor 702. The loudspeaker 790 can be used to communicate out to a user in the service area while the microphone 791 can be used to receive communications from the user in the service area.

The data interface 760 can provide wired data connections, such as one or more universal serial bus (USB) interfaces and/or one or more serial input/output interfaces (e.g., RS232). The data interface 760 can also provide a parallel data interface. The plurality of radio transmitter/receivers (transceivers) 762A-762N can provide wireless data connections such as over WIFI, Bluetooth, and/or cellular. The one or more audio video devices can use the wireless data connections or the wired data connections to communicate with the computer 701.

The computer 701 can be an edge computer that provides for remote logins and remote virtual sessions through one or more of the plurality of network interfaces 761A-761N. Additionally, each of the network interfaces support one or more network connections. Network interfaces can be virtual interfaces and also be logically separated from other virtual interfaces. One or more of the plurality of network interfaces 761A-761N can be used to make network connections between client computers and server computers.

One or more computing systems 700 and/or one or more computers 701 (or computer servers) can be used to perform some or all of the processes disclosed herein. The software instructions that performs the functionality of servers and devices are stored in the storage device 730, 740 and loaded into memory 720 when being executed by the processor 710.

In one embodiment, the processor 710 executes instructions residing on a machine-readable medium, such as the hard disk drive 730, 740, a removable medium (e.g., a compact disk 799, a magnetic tape, etc.), or a combination of both. In an alternate embodiment, one or more of the video interfaces 750A-750B include graphical processing units (GPUs) that can be used to executes instructions and perform functions of the disclosed embodiments. The instructions can be loaded from the machine-readable medium into the memory 720, which can include Random Access Memory (RAM), dynamic RAM (DRAM), etc. The processor 710, 750A-750B can retrieve the instructions from the memory 720 and execute the instructions to perform operations described herein.

Note that any or all of the components and the associated hardware illustrated in FIG. 7 can be used in various embodiments of a computer system 700. However, it should be appreciated that other configurations of the computer system 700 can include more or less devices than those shown in FIG. 7.

### Closing

Some portions of the preceding detailed description have been presented in terms of algorithms and symbolic representations of operations on data bits within a computer memory. These algorithmic descriptions and representations are the tools used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be kept in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The embodiments are thus described. While certain exemplary embodiments have been described and shown in the accompanying drawings, it is to be understood that such embodiments are merely illustrative of and not restrictive on the broad invention, and that the embodiments not be limited to the specific constructions and arrangements shown and described, since various other modifications may occur to those ordinarily skilled in the art.

When implemented in software, the elements of the disclosed embodiments are essentially the code segments to perform the necessary tasks. The program or code segments can be stored in a processor readable medium or transmitted by a computer data signal embodied in a carrier wave over a transmission medium or communication link. The “processor readable medium” may include any medium that can store information. Examples of the processor readable medium include an electronic circuit, a semiconductor memory device, a read only memory (ROM), a flash memory, an erasable programmable read only memory (EPROM), a floppy diskette, a CD-ROM, an optical disk, a hard disk, a fiber optic medium, a radio frequency (RF) link, etc. The computer data signal may include any signal that can propagate over a transmission medium such as electronic network channels, optical fibers, air, electromagnetic, RF links, etc. The code segments may be downloaded using a computer data signal via computer networks such as the Internet, Intranet, etc. and stored in a storage device (processor readable medium).

While this specification includes many specifics, these should not be construed as limitations on the scope of the disclosure or of what may be claimed, but rather as descriptions of features specific to particular implementations of the disclosure. Certain features that are described in this specification in the context of separate implementations may also be implemented in combination in a single implementation. Conversely, various features that are described in the context of a single implementation may also be implemented in multiple implementations, separately or in sub-combination. Moreover, although features may be described above as acting in certain combinations and even initially claimed as such, one or more features from a claimed combination may in some cases be excised from the combination, and the claimed combination may be directed to a sub-combination or variations of a sub-combination. Accordingly, while embodiments have been particularly described, they should not be construed as limited by such disclosed embodiments.

1. A system for training one or more AI models for viral infectivity assays using machine learning, the system comprising;
a first storage device storing one or more captured images captured at a subcellular resolution, each captured image capturing a plurality of stain free cells infected with a known virus stock of a plurality of virus stocks, wherein the plurality of stain free cells include both stain free infected cells and stain free uninfected cells;
a computer system in communication with the first storage device, the computer system including a processor and a second storage device storing instructions for execution by the processor, the processor to execute instructions stored in the second storage device to read and process the one or more captured images stored in the first storage device; and
one or more imaging artificial intelligence (AI) models stored in the second storage device for use by the processor, the one or more imaging AI models to be trained to analyze one or more known viruses, the one or more imaging AI models used with instructions executed by the processor to process the captured images of stain free cells to detect morphological differences between the stain free infected cells and the stain free uninfected cells in the one or more captured images to determine a ratio of stain free infected cells to stain free uninfected cells indicating a predicted viral infectivity based on the one or more captured images,
wherein the morphological differences between the stain free infected cells and the stain free uninfected cells in the one or more captured images are undeterminable by human eyesight at the subcellular resolution.

2. The system of claim 1, wherein
the known virus stock of stain free cells has a known titer of concentration and known ratio of stain free infected cells to stain free uninfected cells.

3. The system of claim 1, wherein the multiplicity of infection (MOI) for the stain free infected cells is greater than 0.999 and the multiplicity of infection (MOI) for the stain free uninfected cells is zero.

4. The system of claim 1, wherein the captured images are captured by an imager to produce images selected from the group of brightfield images, darkfield images, phase contrast images, and differential interference contrast (DIC) images.

5. The system of claim 4, further comprising,
a plate with one or more wells containing the plurality of stain free cells infected with the known virus stock.

6. The system of claim 5, wherein
the one or more captured images are raw images taken of the plate and the one or more wells.

7. The system of claim 6, wherein
the one or more captured images are divided up into non-overlapping rectangular tiles with each tile having M pixels by N pixels numbering in the range inclusively between thirty-two pixels by thirty-two pixels and a pixel width by a pixel height of the one or more captured images.

8. The system of claim 7, wherein
the tiles are prefiltered to reject tiles that are substantially empty of cells; and
the cells are not individually isolated.

9. The system of claim 6, wherein
the one or more captured images are analyzed by the AI model on a cell to cell basis or a tile to tile basis.

10. The system of claim 1, wherein the known virus stock belongs to the family of coronavirus.

11. The system of claim 10, wherein the known virus stock is the SARS-CoV-2 virus.

12. The system of claim 1, further comprises
a database storing metadata associated with the one or more captured images, and
wherein the processor further executes instructions stored in the second storage device to read the stored metadata and to further process the one or more captured images stored in the first storage device based on the stored metadata.

13. A system for analyzing viral infectivity assays using machine learning, the system comprising;
a plate with one or more wells with a plurality of stain free infected cells and a plurality of stain free uninfected cells in each of the one or more wells;
an imager to capture images, at subcellular resolution, of the plate with a plurality of stain free cells infected with a virus stock;
a computer system coupled in communication with the imager, the computer system including a processor and a storage device storing instructions for execution by the processor, wherein the processor when executing the stored instructions in the storage device to provide one or more trained AI models for one or more known viruses, wherein the processor executes further instructions to use the trained AI model to further provides the functionality of analyzing the captured images to determine the ratio of infected to uninfected cells.

14. The system of claim 13, wherein
the plate has a plurality of wells.

15. The system of claim 14, wherein
the plate has a range of one to three wells per sample to increase throughput.

16. The system of claim 13, wherein
the captured images captured by the imager as one selected from the group of brightfield images, darkfield images, phase contrast images, and differential interference contrast (DIC) images.

17. The system of claim 13, wherein
the virus stock contains virus of the group of coated or uncoated DNA and coated or uncoated RNA.

18. The system of claim 17, wherein
the virus is SARS CoV-2, an RNA coated virus which is the causative agent of COVID-19.

19. The system of claim 13, wherein
the imager is a plate imager.

20. The system of claim 13, wherein
the imager is a microscope.

21. The system of claim 13, wherein
the imager is an imaging robot that performs robotic microscopy.

22. The system of claim 13, further comprising
fluid handling robots to process the plates for imaging.

23-70. (canceled)
